(19) United States
(12) Patent Application Publication
(10) Pub. No.: US 2010/0010024 A1
(43) Pub. Date: Jan. 14, 2010

(54) USE OF1,4-DIARYL-DIHYDROPYRIMIDINE-2-ON DERIVATIVES FOR TREATING PULMONARY ARTERIAL HYPERTENSION

(75) Inventors: Franz Von Nussbaum, Dusseldorf (DE); Heike Gielen-Haertwig, Monheim (DE); Martin Klein, Dusseldorf (DE); Volkhart Min-Jian Li, Velbert (DE); Daniel Meibom, Leverkusen (DE); Peter Sandner, Wuppertal (DE); Klemens Lustig, Wuppertal (DE); Stefan Schäfer, Liederbach (DE)

Correspondence Address: Barbara A. Shimei;Director, Patents & Licensing, Bayer HealthCare LLC - Pharmaceuticals, 555 White Plains Road, Third Floor, Tarrytown, NY, 10591, US

(73) Assignee: Bayer Healthcare AG, Leverkusen (DE)

(21) Appl. No.: 12/307,126

(22) PCT Filed: Jun. 25, 2007
(86) PCT No.: PCT/EP2007/005579

§ 371 Date: Sep. 9, 2009

(30) Foreign Application Priority Data
Jul. 1, 2006 (DE) 10 2206 031 314.3

(51) Int. Cl. A61K 31/513; C07D 239/22; C07D 401/04; A61K 31/506; A61P 9/12

(52) U.S. Cl. 514/274; 544/316

(57) ABSTRACT

The present application relates to the use of 1,4-diaryldihydropyrimidin-2-one derivatives of the formula (I) for the treatment and/or prophylaxis of pulmonary arterial hypertension and other types of pulmonary hypertension, and to the use thereof for the manufacture of medicaments for the treatment and/or prophylaxis of pulmonary arterial hypertension and other types of pulmonary hypertension.

The present application relates to the use of 1,4-diaryldihydropyrimidin-2-one derivatives of the formula (I) for the treatment and/or prophylaxis of pulmonary arterial hypertension and other types of pulmonary hypertension, and to the use thereof for the manufacture of medicaments for the treatment and/or prophylaxis of pulmonary arterial hypertension and other types of pulmonary hypertension.

Pulmonary arterial hypertension (PAH) is a progressive lung disorder which, untreated, leads to death on average within 2.8 years after being diagnosed. An increasing constriction of the pulmonary circulation leads to increased stress on the right heart, which may develop into right heart failure. By definition, the mean pulmonary arterial pressure (mPAP) in a case of chronic pulmonary hypertension is >25 mmHg at rest or >30 mmHg during exertion (normal value <20 mmHg). The pathophysiology of pulmonary arterial hypertension is characterized by vasoconstriction and remodeling of the pulmonary vessels. In chronic PAH there is neomuscularization of initially unmuscularized pulmonary vessels, and the vascular muscles of the already muscularized vessels increase in circumference. This increasing obliteration of the pulmonary circulation results in progressive stress on the right heart, which leads to a reduced output from the right heart and eventually ends in right heart failure (M. Humbert et al., J. Am. Coll. Cardiol. 2004, 43, 13S-24S). PAH is an extremely rare disorder, with a prevalence of 1-2 per million. The average age of the patients has been estimated to be 36 years, and only 10% of the patients were over 60 years of age. Distinctly more women than men are affected (G. E. D'Alonzo et al., Ann. Intern. Med. 1991, 115, 343-349).

Despite all the advances in the therapy of pulmonary arterial hypertension there is as yet no prospect of cure of this serious disorder. Standard therapies available on the market (e.g. prostacyclin analogs, endothelin receptor antagonists, phosphodiesterase inhibitors) are able to improve the quality of life, the exercise tolerance and the prognosis of the patients. The principles of these therapies are primarily hemodynamic, influencing vessel tone but having no direct influence on the pathogenic remodeling processes. In addition, the possibility of using these medicaments is restricted through the sometimes serious side effects and/or complicated types of administration. The period over which the clinical situation of the patients can be improved or stabilized by specific monotherapy is limited. Eventually the therapy escalates and thus a combination therapy is applied, where a plurality of medicaments must be given concurrently.

Novel combination therapies are one of the most promising future therapeutic options for the treatment of pulmonary arterial hypertension. In this connection, the finding of novel pharmacological mechanisms for the treatment of PAH is of particular interest (Ghofrani et al., Herz 2005, 30, 296-302; E. B. Rosenzweig, Expert Opin. Emerging Drugs 2006, 11, 609-619; T. Ito et al., Curr. Med. Chem. 2007, 14, 719-733). Therapeutic options which intervene directly in the remodeling event (antiremodeling mechanisms) in particular might form the basis for a more causal treatment and thus be of great advantage for the patients. In this connection, it should be possible to combine known and novel therapies. In order to minimize the risk of interfering medicament-medicament interactions in such a combination therapy, these novel active ingredients ought to inhibit metabolizing P450 CYP enzymes to only a very small extent or not at all.

The term “pulmonary arterial hypertension” includes particular types of pulmonary hypertension as have been specified for example by the World Health Organization (WHO) (Clinical Classification of Pulmonary Hypertension, Venice 2003; G. Simonneau et al., J. Am. Coll. Cardiol. 2004, 43, 5S-12S).

According to this classification, pulmonary arterial hypertension includes idiopathic pulmonary arterial hypertension (IPAH, formerly also called primary pulmonary hypertension), familial pulmonary arterial hypertension (FPAH) and associated pulmonary arterial hypertension (APAH) which is associated with collagenoses, congenital systemic-pulmonary shunts, portal hypertension, HIV infections, intake of particular drugs and medicaments, with other disorders (thyroid disorders, glycogen storage diseases, Gaucher's disease, hereditary teleangiactasia, hemoglobinopathies, myeloproliferative disorders, splenectomy), with disorders with a significant venous/capillary involvement such as pulmonary veno-occlusive disease and pulmonary capillary hemangiomatosis, and persistent pulmonary hypertension of neonates.

Other types of pulmonary hypertension include for example the pulmonary hypertension associated with left heart disorders, e.g. with ventricular or valvular disorders, the pulmonary hypertension associated with disorders of the respiratory tract and/or of the lungs, e.g. with chronic obstructive lung disease, interstitial lung disease or pulmonary fibrosis, the pulmonary hypertension attributable to chronic thrombotic and/or embolic disorders, e.g. associated with thromboembolic obstruction of pulmonary arteries, and the pulmonary hypertension caused by generally inflammatory disease processes or by special causes (e.g. associated with schistosomiasis, sarcoidosis, neoplastic diseases).

Human leukocyte elastase (HLE, EC 3.4.21.37), also called Human Neutrophil Elastase (HNE, hNE), belongs to the family of serine proteases. The proteolytic enzyme is found in the azurophilic granules of polymorphonuclear leukocytes (PMN leukocytes). Intracellular elastase performs an important function in defense against pathogens by breaking down the foreign particles taken by phagocytosis. Activated neutrophilic cells release the HNE from the granules into the extracellular space (extracellular HNE), with some of the released HNE remaining on the outside of the neutrophil cell membrane (membrane-associated HNE). The highly active enzyme is able to break down a large number of connective tissue proteins, e.g. the proteins elastin, collagen and fibronectin. Elastin occurs in high concentrations in all tissue types showing high elasticity, e.g. in the lung and the arteries. HNE is involved in the tissue breakdown and transformation (tissue remodeling) associated with a large number of pathological processes (e.g. tissue injuries). HNE is also an important modulator of inflammatory processes. HNE induces for example an increased interleukin-8 (IL-8) gene expression. It is therefore assumed that HNE plays an important part in many disorders of the lungs (e.g. chronic obstructive pulmonary disease, COPD; acute respiratory distress syndrome, ARDS; cystic fibrosis, CF; lung emphysema) and also in disorders of the cardiovascular system (e.g. tissue changes following a myocardial infarction and associated with heart failure).

It has been possible to find a fragmentation of connective tissue (internal elastic lamina) in animal models and in patients with elevated pulmonary arterial blood pressure (pulmonary arterial hypertension) [Rabinovitch et al., Lab. Invest. 55, 632-653 (1986)]. It was possible to show in animal models of pulmonary arterial hypertension (hypoxic and monocrotaline rat model) that elastase activity was increased and was associated with a fragmentation of connective tissues [Todorovich-Hunter et al., Am. Rev. Respir. Dis. 146, 213-223 (1992)]. It is suspected that the tissue remodeling to be observed during the disease process of pulmonary arterial hypertension is induced by an elastase-mediated release of connective tissue-associated growth factors, e.g. of basic fibroblast growth factor bFGF [Rabinovitch, Am. J. Physiol. 277, L5-L12 (1999)]. It was possible to show a positive effect with an overexpressed elastase inhibitor protein in the hypoxic mouse model of pulmonary arterial hypertension [Zaidi et al., Circulation 105, 516-521 (2002)]. It was possible to show a positive effect with synthetic low molecular weight elastase inhibitors in the monocrotaline rat model of pulmonary arterial hypertension; in this case there was also a beneficial effect on tissue remodeling to be noted [Cowan et al., Nature Med. 6, 698-702 (2000)]. However, all previously disclosed low weight elastase inhibitors have low selectivity, are chemically reactive and/or have only limited oral availability, thus to date thwarting clinical development of an oral elastase inhibitor for these indications.

It is generally assumed that elastase-mediated pathological processes are based on a displaced equilibrium between free elastase and endogenous elastase inhibitor protein (mainly alpha-1 antitryp sin, AAT) [Stockley, Neutrophils and protease/antiprotease imbalance, Am. J. Respir. Crit. Care Med. 160, 49-52 (1999)]. AAT is present in large excess in the plasma and thus very rapidly neutralizes free HNE. The concentration of free elastase is elevated in various pathological processes, so that there is a local shift in the balance between protease and protease inhibitor in favor of the protease. In addition, membrane-associated elastase of the activated PMN-cells is very substantially protected from inhibition by AAT. The same applies to free elastase, which is located in a microcompartment which is difficult to access between the neutrophilic cell and the adjoining tissue cell (e.g. endothelial cell). In addition, strongly oxidizing conditions prevail in the vicinity of activated leukocytes (oxidative burst), and thus AAT is oxidized and loses several orders of magnitude in the inhibitory effect.

Novel elastase-inhibiting active ingredients (exogenously administered inhibitors of HNE) ought accordingly to have a low molecular weight in order to be able also to reach and inhibit the membrane-associated HNE and the HNE present in the protected microcompartment (see above). Also necessary for this purpose is good in vivo stability of the substances (low in vivo clearance). In addition, these compounds ought to be stable under oxidative conditions in order not to lose inhibitory power in the pathological process. For indications in which combination therapies are carried out or are to be expected in future, such as, for example, PAH, it is advantageous in particular that the interaction with enzymes able to transform and break down active pharmaceutical ingredients (P450 CYP enzymes) is only small.

WO 2004/024700, WO 2004/024701, WO 2005/082863 and WO 2005/082864 disclose various 1,4-diaryldihydropyrimidin-2-one derivatives as HNE inhibitors for the treatment of chronic obstructive pulmonary diseases, acute coronary syndrome, myocardial infarction and heart failure.

It has now surprisingly been found that certain 1,4-diaryldihydropyrimidin-2-one derivatives are particularly suitable for the treatment of pulmonary arterial hypertension (PAH). These compounds which are described hereinafter are low molecular weight, unreactive, selective and potent inhibitors of neutrophil elastase which have a sufficiently high bioavailability after oral administration and/or a good solubility for parenteral administration, and show a low in vitro clearance in relation to hepatocytes and only low inhibition of CYP enzymes from microsomes. They thus represent very promising starting points for novel medicaments for the treatment of pulmonary arterial hypertension as monotherapy or in combination with other active ingredients.

The present invention relates to the use of compounds of the general formula (I)

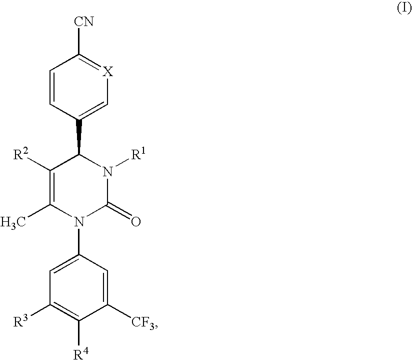

in which

X is CH or N,

R¹ is hydrogen, a group of the formula —(CH₂)ₙ—C(═O)—O—R^{1A} or —(CH₂)ₙ—C(═O)—NR^{1B}R^{1C} or a group of the formula

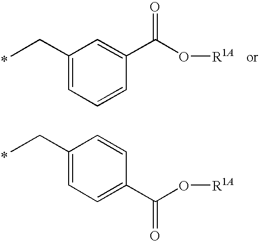

in which
* means the point of linkage to the N atom,
n is the number 1 or 2,
R^{1A} is hydrogen or (C₁-C₄)-alkyl,
and
R^{1B} and R^{1C} are independently of one another hydrogen or (C₁-C₄)-alkyl,

R² is cyano or a group of the formula —C(═O)—R^{2A} or —C(═O)—O—R^{2A}, in which
R^{2A} is (C₁-C₆)-alkyl or (C₃-C₆)-cycloalkyl each of which in turn may be substituted up to twice, identically or differently, by hydroxy, (C₁-C₄)-alkoxy, hydroxycarbonyl, amino, mono- and/or di-(C₁-C₄)-alkylamino, and in which in each case a CH₂ group can be replaced by an O atom as long as a chemically stable compound results,

and

R³ either is hydrogen

and

R⁴ is hydrogen, fluorine or chlorine,

or

R³ is fluorine or chlorine

and

R⁴ is hydrogen,

and the salts, solvates and solvates of the salts thereof for the manufacture of a medicament for the treatment and/or prophylaxis of pulmonary arterial hypertension and other types of pulmonary hypertension.

Preference is given in this connection to the use of compounds of the formula (I) in which

X is CH or N,

R¹ is hydrogen, a group of the formula —CH₂—C(═O)—OH or —CH₂—C(═O)—NH₂ or a group of the formula

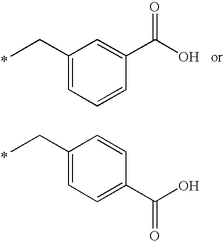

in which
* means the point of linkage to the N atom,

R² is cyano, acetyl, cyclobutylcarbonyl, methoxycarbonyl, ethoxycarbonyl or 2-hydroxy-ethoxycarbonyl,

R³ is hydrogen,

and

R⁴ is hydrogen or fluorine,

and the salts, solvates and solvates of the salts thereof.

Particular preference is given to the use of compounds of formula (I) with the following structures:

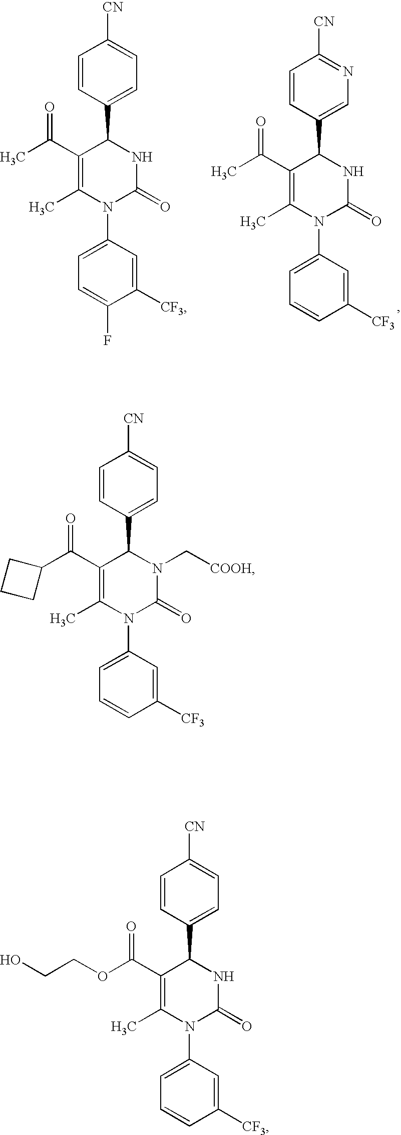

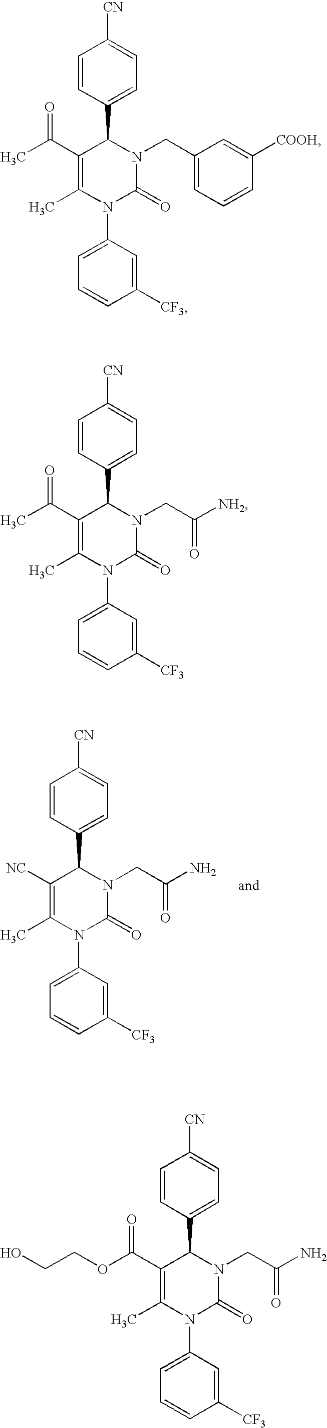

and the salts, solvates and solvates of the salts thereof.

Very particular preference is given to the use of compounds of formula (I) with the following structures:

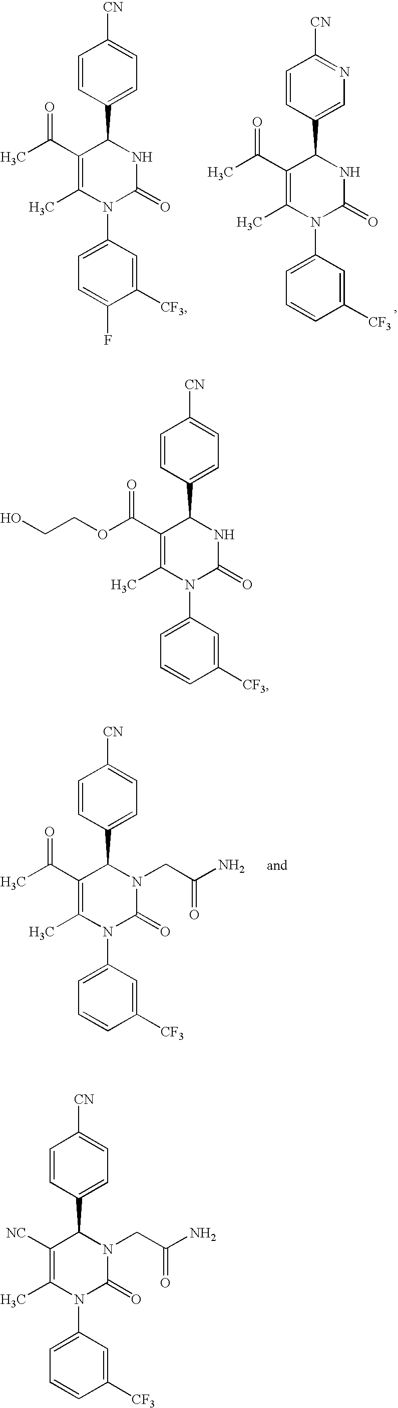

and the salts, solvates and solvates of the salts thereof.

Some of the 1,4-diaryldihydropyrimidin-2-one derivatives of formula (I) are novel as such. The present invention therefore further relates to compounds of formula (I) with the following structures

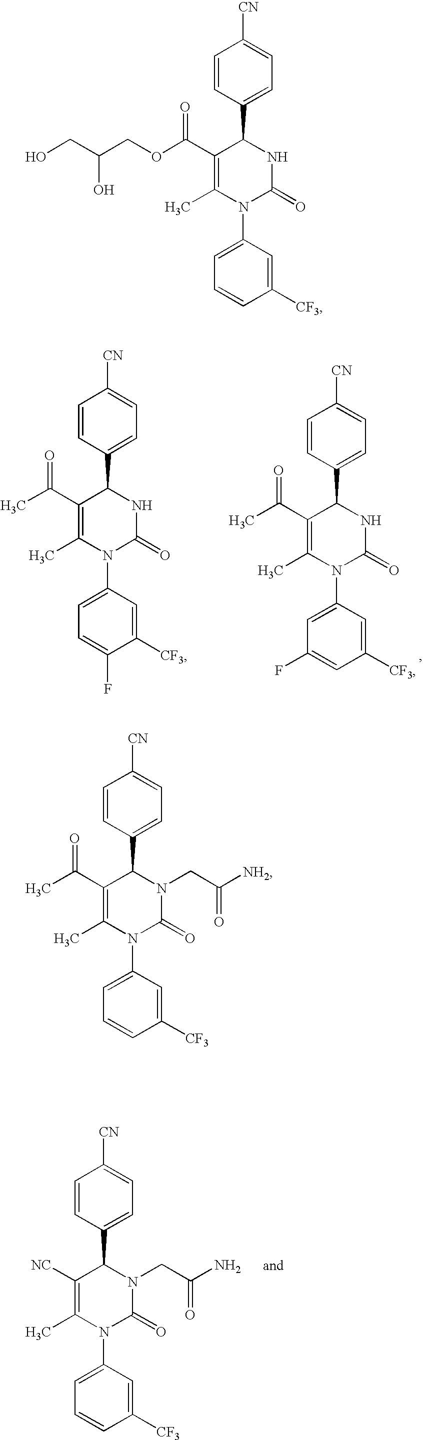

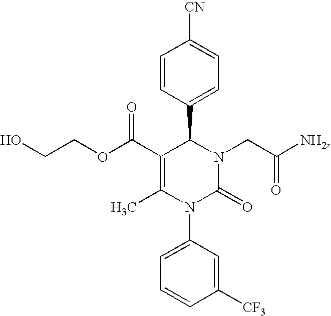

the salts, solvates and solvates of the salts thereof, and the use thereof for the treatment and/or prophylaxis of diseases.

Compounds of the invention, or compounds which can be used according to the invention, also referred to hereinafter comprehensively as compounds of the invention, are the compounds of the formula (I) and the salts, solvates and solvates of the salts thereof, the compounds which are encompassed by formula (I) and are of the formulae mentioned hereinbefore and hereinafter, and the salts, solvates and solvates of the salts thereof, and the compounds which are encompassed by formula (I) and are mentioned hereinafter as exemplary embodiments, and the salts, solvates and solvates of the salts thereof, insofar as the compounds encompassed by formula (I) and mentioned hereinafter are not already salts, solvates and solvates of the salts.

The compounds of the invention may, depending on their structure, exist in stereoisomeric forms (enantiomers, diastereomers). The present invention therefore relates to the enantiomers or diastereomers and respective mixtures thereof. The stereoisomerically pure constituents can be isolated in a known manner from such mixtures of enantiomers and/or diastereomers.

If the compounds of the invention may occur in tautomeric forms, the present invention encompasses all tautomeric forms.

Salts which are preferred for the purposes of the present invention are physiologically acceptable salts of the compounds of the invention. Also encompassed are salts which are themselves unsuitable for pharmaceutical uses but can be used for example for isolating or purifying the compounds of the invention.

Physiologically acceptable salts of the compounds of the invention include acid addition salts of mineral acids, carboxylic acids and sulfonic acids, e.g. salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, trifluoroacetic acid, propionic acid, lactic acid, tartaric acid, malic acid, citric acid, fumaric acid, maleic acid and benzoic acid.

Physiologically acceptable salts of the compounds of the invention include salts of conventional bases such as, by way of example and preferably, alkali metal salts (e.g. sodium and potassium salts), alkaline earth metal salts (e.g. calcium and magnesium salts) and ammonium salts derived from ammonia or organic amines having 1 to 16 C atoms, such as, by way of example and preferably, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

Solvates refers for the purposes of the invention to those forms of the compounds of the invention which form, in the solid or liquid state, a complex by coordination with solvent molecules. Hydrates are a specific form of solvates in which the coordination takes place with water. Hydrates are preferred solvates in the context of the present invention.

The present invention additionally encompasses prodrugs of the compounds of the invention. The term “prodrugs” encompasses compounds which themselves may be biologically active or inactive, but are converted during their residence time in the body into compounds of the invention (for example by metabolism or hydrolysis).

In the context of the present invention, the substituents have the following meaning, unless specified otherwise:

(C₁-C₆)-Alkyl and (C₁-C₄)-alkyl stand for the purposes of the invention for a straight-chain or branched alkyl radical having respectively 1 to 6 and 1 to 4 carbon atoms. A straight-chain or branched alkyl radical having 1 to 4 carbon atoms is preferred. Examples which may be preferably mentioned are: methyl, ethyl, n-propyl, isopropyl, n-butyl, isobutyl, sec-butyl, tert-butyl, 1-ethyl-propyl, n-pentyl and n-hexyl.

(C₃-C₆)-Cycloalkyl stands for the purposes of the present invention for a monocyclic, saturated cycloalkyl group having 3 to 6 carbon atoms. Examples which may be preferably mentioned are: cyclopropyl, cyclobutyl, cyclopentyl and cyclohexyl.

(C₁-C₄)-Alkoxy stands for the purposes of the present invention for a straight-chain or branched alkoxy radical having 1 to 4 carbon atoms. Examples which may be preferably mentioned are: methoxy, ethoxy, n-propoxy, isopropoxy, n-butoxy and tert-butoxy.

Mono-(C₁-C₄)-alkylamino stands for the purposes of the invention for an amino group having a straight-chain or branched alkyl substituent which has 1 to 4 carbon atoms. Examples which may be preferably mentioned are: methylamino, ethylamino, n-propylamino, isopropylamino, n-butylamino and tert-butylamino.

Di-(C₁-C₄)-alkylamino stands for the purposes of the present invention for an amino group with two identical or different straight-chain or branched alkyl substituents each of which have 1 to 4 carbon atoms. Examples which may be preferably mentioned are: N,N-dimethylamino, N,N-diethylamino, N-ethyl-N-methylamino, N-methyl-N-n-propylamino, N-isopropyl-N-methylamino, N-isopropyl-N-n-propylamino, N,N-diisopropylamino, N-n-butyl-N-methylamino and N-tert-butyl-N-methylamino.

The compounds of the invention of the formula (I) are for the most part disclosed in WO 2004/024700, WO 2005/082863 and WO 2005/082864. They can be prepared as described in detail therein or in analogy thereto. In general, the compounds of the formula (I) are prepared by reacting a compound of the formula (II)

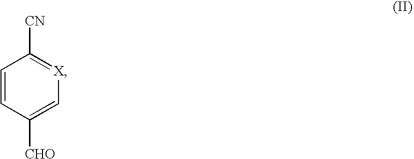

in which X has the meaning indicated above,

in the presence of an acid or of an acid anhydride in a 3-component one-pot reaction or sequentially with a compound of the formula (III)

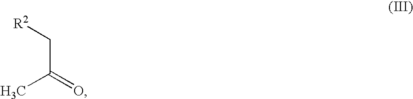

in which R² has the meaning indicated above,

and a compound of the formula (IV)

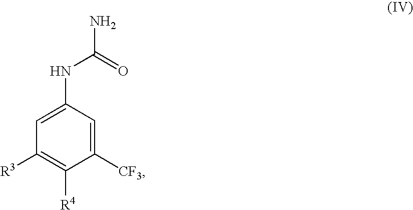

in which R³ and R⁴ have the meanings indicated above,

to give a compound of the formula (I-A)

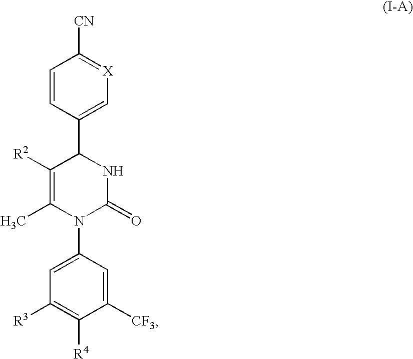

in which R², R³, R⁴ and X each have the meanings indicated above,

and in the case where R¹ is not hydrogen, reacting the latter in the presence of a base with a compound of the formula (V)

R¹*-Z (V),

in which

R¹* has the meaning of R¹ indicated above, but is not hydrogen,

and

Z is a leaving group such as, for example, halogen, mesylate, tosylate or triflate,

and separating the compounds of the formula (I-A) or (I) obtained in this way by methods known to the skilled person into the enantiomers and/or diastereomers thereof and, where appropriate, converting with the appropriate (i) solvents and/or (ii) bases or acids into the solvates, salts and/or solvates of the salts thereof.

Solvents suitable for process step (II)+(III)+(IV)→(I-A) are usual organic solvents which are not altered under the reaction conditions. These include for example ethers such as diethyl ether, diisopropyl ether, methyl tert-butyl ether, 1,2-dimethoxyethane, dioxane or tetrahydrofuran, alcohols such as methanol, ethanol, n-propanol, isopropanol, n-butanol or tert-butanol, hydrocarbons such as pentane, hexane, cyclohexane, benzene, toluene or xylene, halohydrocarbons such as dichloromethane, 1,2-dichloroethane, trichloromethane or chlorobenzene, or other solvents such as ethyl acetate, acetonitrile, dimethyl sulfoxide or N,N-dimethylformamide. It is likewise possible to employ mixtures of the solvents mentioned. Tetrahydrofuran or dioxane is preferably used.

Suitable as acid for process step (II)+(III)+(IV)→(I-A) are usual inorganic or organic acids or acid anhydrides. These include preferably carboxylic acids such as, for example, acetic acid or trifluoroacetic acid, sulfonic acids such as methanesulfonic acid, trifluoromethanesulfonic acid or p-toluenesulfonic acid, hydrochloric acid, sulfuric acid, phosphoric acid, phosphonic acids or phosphoric or phosphonic anhydrides such as polyphosphoric acid, polyphosphoric acid ethyl ester or propanephosphonic anhydride. Polyphosphoric acid ethyl ester is preferably used. The acid is generally employed in an amount of from 0.25 mol to 100 mol based on 1 mol of the compound (III).

Process step (II)+(III)+(IV)→(I-A) is generally carried out in a temperature range from +20° C. to +150° C., preferably at +60° C. to +100° C. The reaction can take place under atmospheric, elevated or reduced pressure (e.g. from 0.5 to 5 bar). It is generally carried out under atmospheric pressure.

Solvents suitable for process step (I-A)+(V)→(I) are usual organic solvents which are not altered under the reaction conditions. These include for example ethers such as diethyl ether, diisopropyl ether, methyl tert-butyl ether, 1,2-dimethoxyethane, dioxane or tetrahydrofuran, hydrocarbons such as pentane, hexane, cyclohexane, benzene, toluene or xylene, halohydrocarbons such as dichloromethane, 1,2-dichloroethane, trichloromethane or chlorobenzene, or other solvents such as ethyl acetate, acetone, methyl ethyl ketone, methyl tert-butyl ketone, acetonitrile, dimethyl sulfoxide, N,N-dimethylformamide, N,N′-dimethylpropyleneurea (DMPU) or N-methylpyrrolidone (NMP). It is likewise possible to employ mixtures of the solvents mentioned. Tetrahydrofuran or dimethyl-formamide is preferably used.

Suitable as base for process step (I-A)+(V)→(I) are usual inorganic or organic bases. These preferably include alkali metal or alkaline earth metal carbonates such as lithium, sodium, potassium, calcium or cesium carbonate, alkali metal alcoholates such as sodium or potassium tert-butoxide, alkali metal hydrides such as sodium or potassium hydride, amides such as lithium or potassium bis(trimethylsilyl)amide or lithiumdiisopropylamide, or organic amines such as triethylamine, N-methylmorpholine, N-methylpiperidine, N,N-diisopropylethylamine, pyridine or 4-N,N-di-methylaminopyridine. Potassium carbonate, cesium carbonate or sodium hydride is preferably used. The base is generally employed in an amount of from 0.1 mol to 10 mol, preferably from 1 mol to 3 mol, based in on 1 mol of the compound (I-A).

Process step (I-A)+(V)→(I) is generally carried out in a temperature range from 0° C. to +150° C., preferably at +20° C. to +80° C. The reaction can take place under atmospheric, elevated or reduced pressure (e.g. from 0.5 to 5 bar). It is generally carried out under atmospheric pressure.

The compounds of the formulae (II), (III), (IV) and (V) are commercially available, known from the literature or can be prepared in analogy to processes known from the literature.

The compounds of the formula (I) which can be used according to the invention can, where appropriate and expedient, also be prepared by transformations of functional groups of individual substituents, in particular those mentioned under R¹ and R², starting from other compounds of the formula (I) obtained by the above process. These transformations are carried out by usual methods and include for example reactions such as esterification, ester cleavage or hydrolysis, reduction, catalytic hydrogenation, oxidation, hydroxylation, amination or alkylation, and the introduction and removal of temporary protective groups.

The process described above can be illustrated by the following reaction schemes:

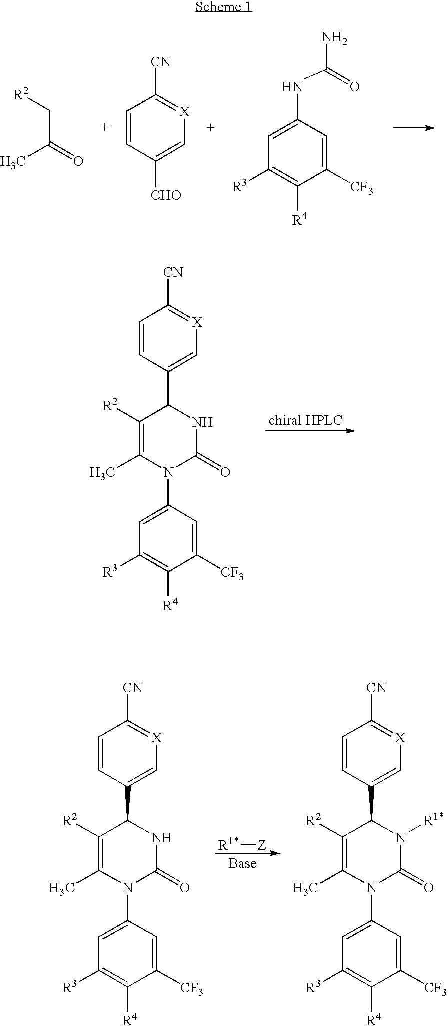

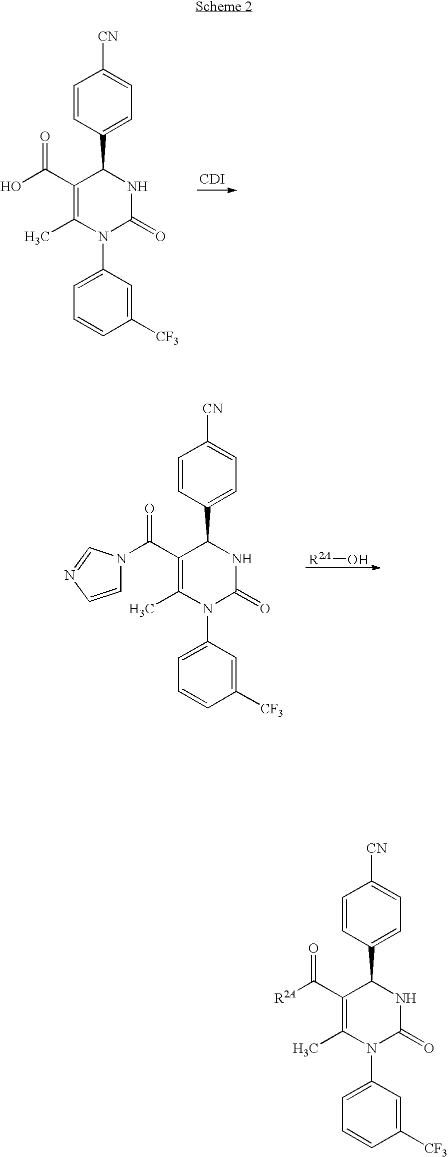

The compounds of the invention are low molecular weight, unreactive, selective and potent inhibitors of neutrophil elastase with expedient physicochemical and pharmacokinetic properties. In particular, their bioavailability after oral administration is sufficiently high and/or their solubility is satisfactory for parenteral administration, and they show a low in vitro clearance in relation to hepatocytes and only low inhibition of CYP enzymes from microsomes.

The compounds of the invention are therefore particularly suitable for the treatment and/or prophylaxis of pulmonary arterial hypertension including its subtypes such as idiopathic and familial pulmonary arterial hypertension, and the pulmonary arterial hypertension which is associated for example with portal hypertension, fibrotic disorders, HIV infection or inappropriate medications or toxins.

The compounds of the invention can also be used for the treatment and/or prophylaxis of other types of pulmonary hypertension. Thus, for example, they can be employed for the treatment and/or prophylaxis of pulmonary hypertension associated with left atrial or left ventricular disorders and with left heart valve disorders. In addition, the compounds of the invention are suitable for the treatment and/or prophylaxis of pulmonary hypertension associated with chronic obstructive pulmonary disease, interstitial pulmonary disease, pulmonary fibrosis, sleep apnoea syndrome, disorders with alveolar hypoventilation, altitude sickness and pulmonary development impairments.

The compounds of the invention are furthermore suitable for the treatment and/or prophylaxis of pulmonary hypertension based on chronic thrombotic and/or embolic disorders such as, for example, thromboembolism of the proximal pulmonary arteries, obstruction of the distal pulmonary arteries and pulmonary embolism. The compounds of the invention can further be used for the treatment and/or prophylaxis of pulmonary hypertension connected with sarcoidosis, histiocytosis X or lymphangioleiomyomatosis, and where the pulmonary hypertension is caused by external compression of vessels (lymph nodes, tumor, fibrosing mediastinitis).

Owing to their pharmacological profile of action, the compounds of the invention are particularly suitable for the treatment and/or prophylaxis of pulmonary arterial hypertension and of pulmonary hypertension associated with chronic obstructive and/or fibrotic pulmonary disorders, and the pulmonary hypertension attributable to chronic thrombotic and/or embolic disorders.

The compounds of the invention can be employed alone or in combination with other active ingredients. The present invention further relates to medicaments comprising at least one of the compounds of the invention and one or more further active ingredients, especially for the treatment and/or prophylaxis of the aforementioned disorders. Suitable active ingredients for combinations are by way of example and preferably:
compounds which inhibit the signal transduction cascade, for example and preferably from the group of kinase inhibitors, in particular from the group of tyrosine kinase and/or serine/threonine kinase inhibitors;
organic nitrates and NO donors such as, for example, sodium nitroprusside, nitroglycerin, isosorbide mononitrate, isosorbide dinitrate, molsidomine or SIN-1, and inhaled NO;
NO-independent but heme-dependent stimulators of soluble guanylate cyclase such as in particular the compounds described in WO 00/06568, WO 00/06569, WO 02/42301 and WO 03/095451;
NO- and heme-independent activators of soluble guanylate cyclase, such as in particular the compounds described in WO 01/19355, WO 01/19776, WO 01/19778, WO 01/19780, WO 02/070462 and WO 02/070510;
prostacycline analogs, such as by way of example and preferably iloprost, beraprost, treprostinil or epoprostenol;
compounds which inhibit soluble epoxide hydrolase (sEH), such as, for example, N,N′-dicyclohexylurea, 12-(3-adamantan-1-yl-ureido)dodecanoic acid or 1-adamantan-1-yl-3-{5-[2-(2-ethoxyethoxy)ethoxy]pentyl}urea;
compounds which influence the energy metabolism of the heart, such as by way of example and preferably etomoxir, dichloroacetate, ranolazine or trimetazidine.
compounds which inhibit the degradation of cyclic guanosine monophosphate (cGMP) and/or cyclic adenosine monophosphate (cAMP), such as, for example, inhibitors of phosphodiesterases (PDE) 1, 2, 3, 4 and/or 5, especially PDE 5 inhibitors such as sildenafil, vardenafil and tadalafil;
agents having an antithrombotic effect, for example and preferably from the group of platelet aggregation inhibitors, of anticoagulants or of profibrinolytic substances;
active ingredients which lower blood pressure, for example and preferably from the group of calcium antagonists, angiotensin All antagonists, ACE inhibitors, endothelin antagonists, renin inhibitors, alpha-receptor blockers, beta-receptor blockers, mineralocorticoid receptor antagonists, Rho kinase inhibitors and diurectics; and/or
active ingredients which alter lipid metabolism, for example and preferably from the group of thyroid receptor agonists, cholesterol synthesis inhibitors such as by way of example and preferably HMG-CoA reductase inhibitors or squalene synthesis inhibitors, of ACAT inhibitors, CETP inhibitors, MTP inhibitors, PPAR-alpha, PPAR-gamma and/or PPAR-delta agonists, cholesterol absorption inhibitors, lipase inhibitors, polymeric bile adsorbents, bile acid reabsorption inhibitors and lipoprotein(a) antagonists.

In a preferred embodiment of the invention, the compounds of the invention are employed in combination with a kinase inhibitor such as by way of example and preferably bortezomib, canertinib, erlotinib, gefitinib, imatinib, lapatinib, lestaurtinib, lonafamib, pegaptinib, pelitinib, semaxanib, sorafenib, sunitinib, tandutinib, tipifamib, vatalanib, fasudil, lonidamine, leflunomide, BMS-3354825 or Y-27632.

Agents having an antithrombotic effect preferably mean compounds from the group of platelet aggregation inhibitors, of anticoagulants or of profibrinolytic substances.

In a preferred embodiment of the invention, the compounds of the invention are administered in combination with a platelet aggregation inhibitor such as by way of example and preferably aspirin, clopidogrel, ticlopidine or dipyridamole.

In a preferred embodiment of the invention, the compounds of the invention are administered in combination with a thrombin inhibitor such as by way of example and preferably ximelagatran, melagatran, bivalirudin or clexane.

In a preferred embodiment of the invention, the compounds of the invention are administered in combination with a GPIIb/IIIa antagonist such as by way of example and preferably tirofiban or abciximab.

In a preferred embodiment of the invention, the compounds of the invention are administered in combination with a factor Xa inhibitor such as by way of example and preferably rivaroxaban, DU-176b, fidexaban, razaxaban, fondaparinux, idraparinux, PMD-3112, YM-150, KFA-1982, EMD-503982, MCM-17, MLN-1021, DX 9065a, DPC 906, JTV 803, SSR-126512 or SSR-128428.

In a preferred embodiment of the invention, the compounds of the invention are administered in combination with heparin or a low molecular weight (LMW) heparin derivative.

In a preferred embodiment of the invention, the compounds of the invention are administered in combination with a vitamin K antagonist such as by way of example and preferably coumarin.

Agents which lower blood pressure preferably mean compounds from the group of calcium antagonists, angiotensin All antagonists, ACE inhibitors, endothelin antagonists, renin inhibitors, alpha-receptor blockers, beta-receptor blockers, mineralocorticoid receptor antagonists, Rho kinase inhibitors, and diuretics.

In a preferred embodiment of the invention, the compounds of the invention are administered in combination with a calcium antagonist such as by way of example and preferably nifedipine, amlodipine, verapamil or diltiazem.

In a preferred embodiment of the invention, the compounds of the invention are administered in combination with an alpha-I receptor blocker such as by way of example and preferably prazosin.

In a preferred embodiment of the invention, the compounds of the invention are administered in combination with a beta-receptor blocker such as by way of example and preferably propranolol, atenolol, timolol, pindolol, alprenolol, oxprenolol, penbutolol, bupranolol, metipranolol, nadolol, mepindolol, carazalol, sotalol, metoprolol, betaxolol, celiprolol, bisoprolol, carteolol, esmolol, labetalol, carvedilol, adaprolol, landiolol, nebivolol, epanolol or bucindolol.

In a preferred embodiment of the invention, the compounds of the invention are administered in combination with an angiotensin All antagonist such as by way of example and preferably losartan, candesartan, valsartan, telmisartan or embusartan.

In a preferred embodiment of the invention, the compounds of the invention are administered in combination with an ACE inhibitor such as by way of example and preferably enalapril, captopril, lisinopril, ramipril, delapril, fosinopril, quinopril, perindopril or trandopril.

In a preferred embodiment of the invention, the compounds of the invention are administered in combination with an endothelin antagonist such as by way of example and preferably bosentan, darusentan, ambrisentan or sitaxsentan.

In a preferred embodiment of the invention, the compounds of the invention are administered in combination with a renin inhibitor such as by way of example and preferably aliskiren, SPP-600 or SPP-800.

In a preferred embodiment of the invention, the compounds of the invention are administered in combination with a mineralocorticoid receptor antagonist such as by way of example and preferably spironolactone or eplerenone.

In a preferred embodiment of the invention, the compounds of the invention are administered in combination with a Rho kinase inhibitor such as by way of example and preferably fasudil, Y-27632, SLx-2119, BF-66851, BF-66852, BF-66853, KI-23095, SB-772077, GSK-269962A or BA-1049.

In a preferred embodiment of the invention, the compounds of the invention are administered in combination with a diuretic such as by way of example and preferably furosemide.

Agents which alter lipid metabolism preferably mean compounds from the group of CETP inhibitors, thyroid receptor agonists, cholesterol synthesis inhibitors such as HMG-CoA reductase inhibitors or squalene synthesis inhibitors, of ACAT inhibitors, MTP inhibitors, PPAR-alpha, PPAR-gamma and/or PPAR-delta agonists, cholesterol absorption inhibitors, polymeric bile acid adsorbents, bile acid reabsorption inhibitors, lipase inhibitors and lipoprotein(a) antagonists.

In a preferred embodiment of the invention, the compounds of the invention are administered in combination with a CETP inhibitor such as by way of example and preferably torcetrapib (CP-529 414), JJT-705 or CETP vaccine (Avant).

In a preferred embodiment of the invention, the compounds of the invention are administered in combination with a thyroid receptor agonist such as by way of example and preferably D-thyroxine, 3,5,3′-triiodothyronine (T3), CGS 23425 or axitirome (CGS 26214).

In a preferred embodiment of the invention, the compounds of the invention are administered in combination with an HMG-CoA reductase inhibitor from the class of statins such as by way of example and preferably lovastatin, simvastatin, pravastatin, fluvastatin, atorvastatin, rosuvastatin, cerivastatin or pitavastatin.

In a preferred embodiment of the invention, the compounds of the invention are administered in combination with a squalene synthesis inhibitor such as by way of example and preferably BMS-188494 or TAK-475.

In a preferred embodiment of the invention, the compounds of the invention are administered in combination with an ACAT inhibitor such as by way of example and preferably avasimibe, melinamide, pactimibe, eflucimibe or SMP-797.

In a preferred embodiment of the invention, the compounds of the invention are administered in combination with an MTP inhibitor such as by way of example and preferably implitapide, BMS-201038, R-103757 or JTT-130.

In a preferred embodiment of the invention, the compounds of the invention are administered in combination with a PPAR-gamma agonist such as by way of example and preferably pioglitazone or rosiglitazone.

In a preferred embodiment of the invention, the compounds of the invention are administered in combination with a PPAR-delta agonist such as by way of example and preferably GW-501516 or BAY 68-5042.

In a preferred embodiment of the invention, the compounds of the invention are administered in combination with a cholesterol absorption inhibitor such as by way of example and preferably ezetimibe, tiqueside or pamaqueside.

In a preferred embodiment of the invention, the compounds of the invention are administered in combination with a lipase inhibitor such as by way of example and preferably orlistat.

In a preferred embodiment of the invention, the compounds of the invention are administered in combination with a polymeric bile adsorbent such as by way of example and preferably cholestyramine, colestipol, colesolvam, CholestaGel or colestimide.

In a preferred embodiment of the invention, the compounds of the invention are administered in combination with a bile acid reabsorption inhibitor such as by way of example and preferably ASBT (=IBAT) inhibitors such as, for example, AZD-7806, S-8921, AK-105, BARI-1741, SC-435 or SC-635.

In a preferred embodiment of the invention, the compounds of the invention are administered in combination with a lipoprotein(a) antagonist such as by way of example and preferably gemcabene calcium (CI-1027) or nicotinic acid.

The present invention further relates to the use of the compounds of the invention, alone or in combination with one or more of the aforementioned active ingredients, for the manufacture of a medicament for the treatment and/or prophylaxis of idiopathic or familial pulmonary arterial hypertension, or pulmonary arterial hypertension associated with medicaments, toxins or other disorders, for the treatment and/or prophylaxis of pulmonary hypertension associated with left atrial or left ventricular disorders, left heart valve disorders, chronic obstructive pulmonary disease, interstitial pulmonary disease, pulmonary fibrosis, sleep apnoea syndrome, disorders with alveolar hypoventilation, altitude sickness, pulmonary development impairments, chronic thrombotic and/or embolic disorders such as, for example, thromboembolism of the proximal pulmonary arteries, obstruction of the distal pulmonary arteries and pulmonary embolism, or in conjunction with sarcoidosis, histiocytosis X or lymphangioleiomyomatosis, and for the treatment and/or prophylaxis of pulmonary hypertension caused by external compression of vessels.

The present invention further relates to a method for the treatment and/or prophylaxis of pulmonary arterial hypertension and other types of pulmonary hypertension in humans and animals by administering an effective amount of at least one of the compounds of the invention or of a medicament comprising at least one of the compounds of the invention.

The medicaments to be manufactured in accordance with the use according to the invention or to be used according to the invention comprise at least one of the compounds of the invention, normally together with one or more inert, non-toxic, pharmaceutically suitable excipients.

The present invention further relates to medicaments comprising at least one of the compounds of the invention in combination with one or more inert, non-toxic, pharmaceutically suitable excipients for the treatment and/or prophylaxis of the aforementioned disorders.

The compounds of the invention may have systemic and/or local effects. For this purpose, they can be administered in a suitable way such as, for example, by the oral, parenteral, pulmonary, nasal, sublingual, lingual, buccal, rectal, dermal, transdermal, conjunctival or otic route or as implant or stent.

The compounds of the invention can be administered in administration forms suitable for these administration routes.

Suitable for oral administration are administration forms which function according to the prior art and deliver the compounds of the invention rapidly and/or in a modified manner, and which contain the compounds of the invention in crystalline and/or amorphized and/or dissolved form, such as, for example, tablets (uncoated and coated tablets, for example having coatings which are resistant to gastric juice or are insoluble or dissolve with a delay and control the release of the compound of the invention), tablets which disintegrate rapidly in the mouth, or films/wafers, films/lyophilizates, capsules (for example hard or soft gelatin capsules), sugar-coated tablets, granules, pellets, powders, emulsions, suspensions, aerosols or solutions.

Parenteral administration can take place with avoidance of an absorption step (e.g. intravenous, intraarterial, intracardiac, intraspinal or intralumbar) or with inclusion of an absorption (e.g intramuscular, subcutaneous, intracutaneous, percutaneous, or intraperitoneal). Administration forms suitable for parenteral administration are, inter alia, preparations for injection and infusion in the form of solutions, suspensions, emulsions, lyophilizates or sterile powders.

Suitable for the other routes of administration are, for example, pharmaceutical forms for inhalation (inter alia powder inhalers, nebulizers), nasal drops, solutions or sprays; tablets for lingual, sublingual or buccal administration, films/wafers or capsules, suppositories, preparations for the ears and eyes, vaginal capsules, aqueous suspensions (lotions, shaking mixtures), lipophilic suspensions, ointments, creams, transdermal therapeutic systems (for example patches), milk, pastes, foams, dusting powders, implants or stents.

Oral or parenteral administration are preferred, especially oral and intravenous administration.

The compounds of the invention can be converted into the stated administration forms. This can take place in a manner known per se by mixing with inert, non-toxic, pharmaceutically suitable excipients. These excipients include inter alia carriers (for example microcrystalline cellulose, lactose, mannitol), solvents (e.g. liquid polyethylene glycols), emulsifiers and dispersants or wetting agents (for example sodium dodecyl sulfate, polyoxysorbitan oleate), binders (for example polyvinylpyrrolidone), synthetic and natural polymers (for example albumin), stabilizers (e.g. antioxidants such as, for example, ascorbic acid), colorings (e.g. inorganic pigments such as, for example, iron oxides) and masking flavors and/or odors.

It has generally proved to be advantageous on parenteral administration to administer amounts of about 0.001 to 1 mg/kg, preferably about 0.01 to 0.5 mg/kg of body weight per day to achieve effective results. On oral administration, the dosage is about 0.01 to 100 mg/kg, preferably about 0.01 to 20 mg/kg, and very particularly preferably 0.1 to 10 mg/kg of body weight.

It may nevertheless be necessary where appropriate to deviate from the stated amounts, in particular as a function of body weight, administration route, individual response to the active ingredient, type of preparation and time or interval over which administration takes place. Thus, in some cases it may be sufficient to make do with less than the aforementioned minimum amount, whereas in other cases the upper limit mentioned must be exceeded. Where relatively large amounts are administered, it may be advisable to distribute these in a plurality of single doses over the day.

The following exemplary embodiments illustrate the invention. The invention is not restricted to the examples.

The percentage data in the following tests and examples are, unless indicated otherwise, percentages by weight; parts are parts by weight. Solvent ratios, dilution ratios and concentration data of liquid/liquid solutions are, unless indicated otherwise, based in each case on the volume.

### A. EXAMPLES

Abbreviations:

aq. aqueous, aqueous solution

cat. catalytic

CDI N,N′-Carbonyldiimidazole

DCI direct chemical ionization (in MS)

DMF dimethylfonnamide

DMSO dimethyl sulfoxide

ee enantiomeric excess

eq. equivalent(s)

ESI electrospray ionization (in MS)

h hour(s)

HATU O-(7-Azabenzotriazol-1-yl)-N,N,N′,N′-tetramethyluronium hexafluorophosphate

HPLC high-pressure, high-performance liquid chromatography

LC-MS coupled liquid chromatography-mass spectrometry

min minute(s)

MPLC medium pressure liquid chromatography

MS mass spectrometry

NMR nuclear magnetic resonance spectrometry

RT room temperature

Rₜ retention time (in HPLC)

TFA trifluoroacetic acid

THF tetrahydrofuran

TLC thin-layer chromatography

UV ultraviolet spectrometry

v/v volume to volume ratio (of a solution)

HPLC and LC-MS Methods:

Method 1 (Analytical HPLC):

HPLC instrument type: HP 1100 series; UV DAD; column: Phenomenex Synergi 2μ Hydro-RP Mercury 20 mm×4 mm; eluent A: 1 l water+0.5 ml 50% formic acid, eluent B: 1 l acetonitrile+0.5 ml 50% formic acid; gradient: 0.0 min 90% A→2.5 min 30% A→3.0 min 5% A→4.5 min 5% A; flow rate: 0.0 min 1 ml/min→2.5 min/3.0 min/4.5 min 2 ml/min; oven: 50° C.; UV detection: 210nm.

Method 2 (Analytical HPLC):

HPLC instrument type: HP 1050 series; UV DAD; column: Phenomenex Synergi 2μ Max-RP Mercury 20 mm×4 mm; eluent A: 1 l water+0.05% trifluoroacetic acid, eluent B: 1 l acetonitrile; gradient: 0.0 min 90% A→2.5 min 30% A→3.0 min 5% A→4.5 min 5% A; flow rate: 0.0 min 1 ml/min→2.5 min/3.0 min/4.5 min 2 ml/min; oven: 50° C.; UV detection: 210 nm.

Method 3 (Analytical HPLC):

Instrument: HP 1100 with DAD detection; column: Kromasil 100 RP-18, 60 mm×2.1 mm, 3.5 μm; eluent A: 5 ml HClO₄ (70%)/L water, eluent B: acetonitrile; gradient: 0 min 2% B→0.5 min 2% B→4.5 min 90% B→6.5 min 90% B→6.7 min 2% B→7.5 min 2% B; flow rate: 0.75 ml/min; column temperature: 30° C.; UV detection: 210 nm.

Method 4 (Analytical HPLC):

Instrument: HP 1100 with DAD detection; column: Kromasil 100 RP-18, 60 mm×2.1 mm, 3.5 μm; eluent A: 5 ml HClO₄ (70%)/L water, eluent B: acetonitrile; gradient: 0 min 2% B→0.5 min 2% B→4.5 min 90% B→9 min 90% B→9.2 min 2% B→10 min 2% B; flow rate: 0.75 ml/min; column temperature: 30° C.; UV detection: 210 nm.

Method 5 (Analytical HPLC on a Chiral Phase):

Chiral silica gel phase based on the selector poly(N-methacryloyl-D-leucine tert-butylamide; column: 250 mm×4.6 mm; eluent: ethyl acetate; flow rate: 2.0 ml/min; temperature: 24° C.; UV detection: 260 nm.

Method 6 (LC-MS):

MS instrument type: Micromass ZQ; HPLC instrument type: Waters Alliance 2795/HP 1100; column: Phenomenex Synergi 2μ Hydro-RP Mercury 20 mm×4 mm; eluent A: 1 l water+0.5 ml 50% formic acid, eluent B: 1 l acetonitrile+0.5 ml 50% formic acid; gradient: 0.0 min 90% A→2.5 min 30% A→3.0 min 5% A→4.5 min 5% A; flow rate: 0.0 min 1 ml/min→2.5 min/3.0 min/4.5 min 2 ml/min; oven: 50° C.; UV detection: 210 nm.

Method 7 (LC-MS):

MS instrument type: Micromass ZQ; HPLC instrument type: Waters Alliance 2795; column: Phenomenex Synergi 2μ Hydro-RP Mercury 20 mm×4 mm; eluent A: 1 l water+0.5 ml 50% formic acid, eluent B: 1 l acetonitrile+0.5 ml 50% formic acid; gradient: 0.0 min 90% A→2.5 m 30% A→3.0 min 5% A→4.5 min 5% A; flow rate: 0.0 min 1 ml/min→2.5 min/3.0 min/4.5 min 2 ml/min; oven: 50° C.; UV detection: 210 nm.

Method 8 (LC-MS):

Instrument: Micromass Quattro LCZ with HPLC Agilent series 1100; column: Phenomenex Synergi 2μ Hydro-RP Mercury 20 mm×4 mm; eluent A: 1 l water+0.5 ml 50% formic acid, eluent B: 1 l acetonitrile+0.5 ml 50% formic acid; gradient: 0.0 min 90% A→2.5 min 30% A→3.0 min 5% A→4.5 min 5% A; flow rate: 0.0 min 1 ml/min→2.5 min/3.0 min/4.5 min 2 ml/min; oven: 50° C.; UV detection: 208-400 nm.

Method 9 (LC-MS):

Instrument: Micromass Platform LCZ with HPLC Agilent series 1100; column: Thermo HyPURITY Aquastar 3μ 50 mm×2.1 mm; eluent A: 1 l water+0.5 ml 50% formic acid, eluent B: 1 l acetonitrile+0.5 ml 50% formic acid; gradient: 0.0 min 100% A→0.2 min 100% A→2.9 min 30% A→3.1 min 10% A→5.5 min 10% A; oven: 50° C.; flow rate: 0.8 ml/min; UV detection: 210 nm.

Method 10 (LC-MS):

MS instrument type: Micromass ZQ; HPLC instrument type: HP 1100 series; UV DAD; column: Phenomenex Gemini 3μ 30 mm×3.0 mm; eluent A: 1 l water+0.5 ml 50% formic acid, eluent B: 1 l acetonitrile+0.5 ml 50% formic acid; gradient: 0.0 min 90% A→2.5 min 30% A→3.0 min 5% A→4.5 min 5% A; flow rate: 0.0 min 1 ml/min→2.5 min/3.0 min/4.5 min 2 ml/min; oven: 50° C.; UV detection: 210 nm.

Method 11 (Preparative HPLC):

Instrument: Gilson Abimed HPLC; binary pump system; column: Kromasil-100A C18, 5 μm, 250 mm×21 mm; eluent A: water/0.5% trifluoroacetic acid, eluent B: acetonitrile; 0-10 min 10% B, ramp 10.01-55 min 100% B; flow rate: 20 ml/min; UV detection: 210 nm.

Method 12 (Preparative HPLC):

Instrument: Gilson Abimed HPLC; binary pump system; column: Kromasil-100A C18, 5 μm, 250 mm×20 mm; eluent A: water/0.05% trifluoroacetic acid, eluent B: acetonitrile; 0-1 min 10% B, ramp 1.01-30 min 95% B, 30.01-34 min 95% B, 34.01-35 min 10% B; flow rate: 25 ml/min; UV detection: 210 nm.

Method 13 (Preparative HPLC):

Instrument: Gilson Abimed HPLC; binary pump system; column: Kromasil-100A C18, 5 μm, 250 mm×20 mm; eluent A: water/0.1% trifluoroacetic acid, eluent B: acetonitrile; 0-1 min 10% B, ramp 1.01-40 min 90% B, 40-45 min 90% B; flow rate: 25 ml/min; UV detection: 210 nm.

Method 14 (Preparative HPLC):

Instrument: Gilson Abimed HPLC; binary pump system; column: GromSil 120 ODS-4HE, 250 mm×40 mm, 10 μm; eluent A: water, eluent B: acetonitrile; 0-3 min 10% B, ramp 3.01-27 min 98% B, 27.01-34 min 98% B, 34.01-38 min 10% B; flow rate: 50 ml/min; UV detection: 214 nm.

Method 15 (Preparative HPLC):

Instrument: Abimed Gilson Pump 305/306, Manometric Module 806; column: GromSil 120 ODS-4HE, 10 μm, 250 mm×30 mm; eluent A: water, eluent B: acetonitrile; gradient: 0.0 min 30% B→3 min 30% B→31 min 95% B→44 min 95% B→45 min 30% B; flow rate: 50 ml/min; column temperature: RT; UV detection: 210 nm.

Method 16 (Preparative HPLC on a Chiral Phase):

Enantiomer separation on a chiral silica gel phase based on the selector poly(N-methacryloyl-L-leucine tert-butylamide); column: 125 mm×20 mm; the sample is dissolved in a 1:5 THF/ethyl acetate mixture; eluent: ethyl acetate; flow rate: 20 ml/min; UV detection: 260 nm; temperature: 24° C.

Method 17 (Preparative HPLC on a Chiral Phase):

Enantiomer separation on a chiral silica gel phase based on the selector poly(N-methacryloyl-L-leucine D-menthylamide); column: 250 mm x 30 mm; eluent: step gradient 100% ethyl acetate→100% methanol; flow rate: 50 ml/min; temperature: 24° C.; UV detection: 260 nm. Analytical column: 250 mm×4.6 mm; eluent: ethyl acetate/methanol 10:1; flow rate: 2 ml/min.

Method 18 (Preparative HPLC on a Chiral Phase):

Enantiomer separation on a chiral silica gel phase based on the selector poly(N-methacryloyl-L-leucine 1-menthylamide); column: 680 mm×40 mm; eluent: step gradient 100% ethyl acetate→100% methanol; flow rate: 50 ml/min; temperature: 24° C.; UV detection: 260 nm. Analytical column: 250 mm×4.6 mm; eluent: ethyl acetate; flow rate: 2 ml/min.

Method 19 (Preparative HPLC on a Chiral Phase):

Enantiomer separation on a chiral silica gel phase based on the selector poly(N-methacryloyl-L-leucin D-menthylamide); column: 250 mm×30 mm; eluent: step gradient 100% ethyl acetate→100% methanol; flow rate: 30 mumin; temperature: 24° C.; UV detection: 260 nm. Analytical column: 250 mm×4.6 mm; eluent: ethyl acetate/methanol 5:1; flow rate: 2 ml/min.

Method 20 (Preparative HPLC on a Chiral Phase):

Enantiomer separation on a chiral silica gel phase based on the selector poly(N-methacryloyl-D-leucine 3-pentylamide; column: 500 mm×63 mm; eluent: step gradient 100% ethyl acetate (0-16.33 min)→100% methanol (16.34-24.12 min)→100% ethyl acetate (24.13-35.0 min); flow rate: 100 ml/min; temperature: 24° C.; UV detection: 340 nm.

Method 21 (Preparative HPLC on a Chiral Phase):

Enantiomer separation on a chiral silica gel phase based on the selector poly(N-methacryloyl-L-leucine dicyclopropylmethylamide); column: 250 mm×20 mm; eluent: step gradient isohexane/ethyl acetate (40:60) (0-8 min)→100% ethyl acetate (8.01-12 min)→isohexane/ethyl acetate (40:60) (12.01-20 min); flow rate: 25 ml/min; temperature: 24° C.; UV detection: 280 nm. Analytical column: 250 mm×4.6 mm; eluent: ethyl acetate/isohexane 1:1; flow rate: 2 ml/min.

Method 22 (Preparative HPLC):

Instrument: Gilson Abimed HPLC; binary pump system; column: GromSil 120 ODS-4HE, 250 mm×40 mm, 10 μm; eluent A: water+0.1% trifluoroacetic acid, eluent B: acetonitrile; 10% B, ramp to 90% B in 45 min.

Method 23 (Preparative HPLC):

Instrument: Abimed Gilson Pump 305/306, Manometric Module 806; column: GromSil C18, 250 mm×30 mm, 10 μm; eluent A: water+0.1% trifluoroacetic acid, eluent B: acetonitrile; gradient: 0-3 min 10% B, ramp 3.01-34 min 95% B, 34.01-38 min 95% B, 38.01-40 min 10% B; flow rate: 50 ml/min; UV detection: 210 nm.

Method 24 (LC-MS):

MS instrument type: Waters ZQ; HPLC instrument type: Waters Alliance 2795; column: Phenomenex Onyx Monolithic C 18, 100 mm×3 mm; eluent A: 1 l water+0.5 ml 50% formic acid, eluent B: 1 l acetonitrile+0.5 ml 50% formic acid; gradient: 0.0 min 90% A→2 min 65% A→4.5 min 5% A→6 min 5% A; flow rate: 2 ml/min; oven: 40° C.; UV detection: 210 nm.

Method 25 (LC-MS):

Instrument: Micromass Quattro LCZ with HPLC Agilent Series 1100; column: Phenomenex Onyx Monolithic C18, 100 mm×3 mm; eluent A: 1 l water+0.5 ml 50% formic acid, eluent B: 1 l acetonitrile+0.5 ml 50% formic acid; gradient: 0.0 min 90% A→2 min 65% A→4.5 min 5% A→6 min 5% A; flow rate: 2 ml/min; oven: 40° C.; UV detection: 208-400 nm.

Starting Compounds and Intermediates:

### Example 1A

### N-[3-Fluoro-5-(trifluoromethyl)phenyl]urea

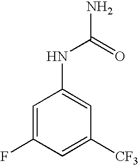

34 g (189.819 mmol) of 5-fluoro-3-(trifluoromethyl)aniline are dissolved in 227 ml of 2-propanol and, at 50° C., 32.803 g (284.728 mmol) of trimethylsilyl isocyanate are added dropwise over a period of 10 minutes. The mixture is stirred at 50° C. overnight and then concentrated in a rotary evaporator. The residue is stirred with dichloromethane, and the solid is filtered off with suction and dried under high vacuum. 25.0 g (59% of theory) of the target compound are obtained.

LC-MS (method 7): Rₜ=1.69 min; MS (ESIpos): m/z (%)=223.0 (100) [M+H]⁺

HPLC (method 3): Rₜ=3.88 min

¹H-NMR (400 MHz, DMSO-d6): δ=6.12 (br. s, 2H), 7.12 (d, 1H), 7.53 (d, 1H), 7.61 (s, 1H), 9.12 (br. s, 1H).

### Example 2A

### N-[4-Fluoro-3-(trifluoromethyl)phenyl]urea

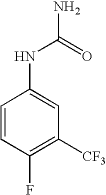

2500 mg (13.957 mmol) of 4-fluoro-3-(trifluoromethyl)aniline are dissolved in 15 ml of 1 N hydrochloric acid, and 1132 mg (13.957 mmol) of potassium cyanate are added. The suspension is stirred at room temperature overnight and then diluted with ethyl acetate to obtain a clear two-phase solution. The organic phase is separated off and the aqueous is extracted with ethyl acetate. After the combined organic phases have been dried and the solvent has been stripped off in a rotary evaporator, the crude product is chromatographed on silica gel (eluent: dichloromethane/methanol 80:1, then 10:1). 2180 mg (70% of theory) of the target compound are obtained.

LC-MS (method 10): Rₜ=1.82 min; MS (ESIpos): m/z (%)=223.0 (100) [M+H]⁺.

### Example 3A

### 4-{(4R)-5-(1H-Imidazol-1-ylcarbonyl)-6-methyl-2-oxo-1-[3-(trifluoromethyl)phenyl]-1,2,3,4-tetrahydropyrimidin-4-yl}benzonitrile

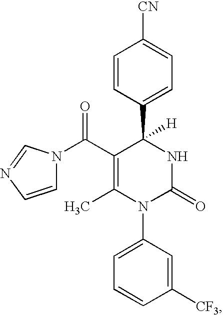

Under an argon protective gas atmosphere, (4R)-4-(4-cyanophenyl)-6-methyl-2-oxo-1-[3-(trifluoromethyl)phenyl]-1,2,3,4-tetrahydropyrimidine-5-carboxylic acid (2.05 g, 5 mmol; preparation according to WO 2005/082863, Example 10A) is introduced into dry DMF (21 ml), and 1,1′-carbonyldiimidazole (2.43 g, 15 mmol) is added. The mixture is stirred at room temperature for 30 min. It is then concentrated, taken up in ethyl acetate (about 150 ml), washed with saturated aqueous sodium bicarbonate solution (about 50 ml) and saturated aqueous sodium chloride solution (50 ml), dried over sodium sulfate, filtered and concentrated in vacuo. The title compound is obtained as a crude product (2.87 g, quantitative yield, purity about 80%), which is reacted without further purification.

HPLC (method 2): Rₜ=2.3 min.

### Example 4A

### (4R)-3-(2-tert-Butoxy-2-oxoethyl)-4-(4-cyanophenyl)-6-methyl-2-oxo-1-[3-(trifluoromethyl)phenyl]-1,2,3,4-tetrahydropyrimidine-5-carboxylic acid

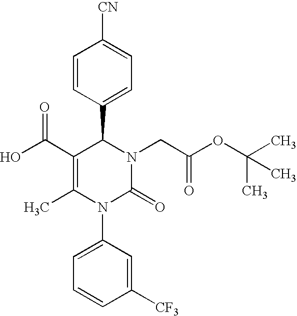

The reaction is carried out under argon. Allyl(4R)-3-(2-tert-butoxy-2-oxoethyl)-4-(4-cyanophenyl)-6-methyl-2-oxo-1-[3-(trifluoromethyl)phenyl]-1,2,3,4-tetrahydropyrimidine-5-carboxylate (2.50 g, 4.5 mmol; for the preparation of the racemic compound, see WO 2005/082863, Example 23A) and morpholine (588 mg, 6.8 mmol) are introduced into THF (25 ml). The mixture is cautiously evacuated and again flushed with argon. Then tetrakis(triphenylphosphine)palladium(0) (260 mg, 0.225 mmol) is added, and the reaction mixture is stirred at RT for 60 min. An HPLC check then shows complete conversion. The reaction mixture is concentrated in vacuo, and the residue is taken up in ethyl acetate (200 ml). The organic phase is then washed with saturated ammonium chloride solution (75 ml), water (50 ml) and saturated sodium chloride solution. The organic phase is dried over sodium sulfate, filtered and concentrated, and the residue is purified by preparative HPLC (column: Kromasil 5 μm; eluent: acetonitrile/water+0.1% TFA 10:90→90:10). The title compound is obtained in this way as a solid (1.9 g, 82% of theory).

LC-MS (method 7): Rₜ=2.4 min; MS (ESIpos): m/z (%)=516 (5) [M+H]⁺; MS (ESIneg): m/z (%)=514.2 (100) [M−H]⁻.

### Exemplary Embodiments

### Example 1

### Ethyl(4R)-4-(4-cyanophenyl)-6-methyl-2-oxo-1-[3-(trifluoromethyl)phenyl]-1,2,3,4-tetrahydropyrimidine-5-carboxylate

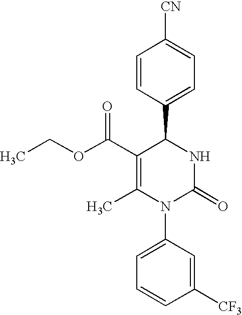

Racemic ethyl 4-(4-cyanophenyl)-6-methyl-2-oxo-1-[3-(trifluoromethyl)phenyl]-1,2,3,4-tetrahydropyrimidine-5-carboxylate (60 g; for preparation, see WO 2004/024700, Example 1) is dissolved in ethyl acetate (360 ml) and fractionated into the enantiomers by chromatography (method 20). The title compound (29.9 g, 99% of theory, 99.1% ee) and the isomeric ethyl(4S)-4-(4-cyanophenyl)-6-methyl-2-oxo-1-[3-(trifluoromethyl)phenyl]-1,2,3,4-tetrahydropyrimidine-5-carboxylate are obtained.

HPLC (method 5): Rₜ=1.55 min.

The further analytical data correspond to those reported for the racemic compound (see WO 2004/024700, Example 1).

### Example 2

### 4-{(4R)-5-Isobutyryl-6-methyl-2-oxo-1-[3-(trifluoromethyl)phenyl]-1,2,3,4-tetrahydropyrimidin-4-yl}benzonitrile

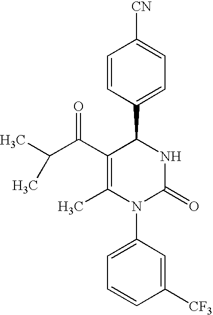

2000 mg (15.604 mmol) of 5-methylhexane-2,4-dione, 2046 mg (15.604 mmol) of 4-formyl-benzonitrile and 3186 mg (15.604 mmol) of 1-[3-(trifluoromethyl)phenyl]urea are dissolved in 60 ml of THF, and 10 g of ethyl polyphosphate (PPE) are added. The mixture is stirred at 80° C. overnight and then partitioned between water and ethyl acetate. The organic phase is separated off, dried over sodium sulfate and filtered, and the solvent is stripped off in a rotary evaporator. The residue is purified firstly by flash chromatography on silica gel (eluent: cyclohexane/ethyl acetate 1:1) and then by preparative HPLC on a chiral phase (method 18), thus separating the enantiomers (desired enantiomer: Rₜ=4.70 min). Further purification by preparative HPLC (method 15) results in 530 mg (8% of theory) of the title compound.

MS (ESIpos): m/z (%)=428 (100) [M+H]⁺

HPLC (method 3): Rₜ=8.33 min

¹H-NMR (400 MHz, DMSO-d₆): δ=0.80 (d, 3H), 0.95 (d, 3H), 1.86 (s, 3H), 2.95 (tt, 1H), 5.48 (d, 1H), 7.55 (d, 1H), 7.62 (d, 2H), 7.68 (t, 1H), 7.70 (s, 1H), 7.77 (d, 1H), 7.89 (d, 2H), 8.41 (d, 1H).

### Example 3

### 4-{(4R)-5-(Cyclobutylcarbonyl)-6-methyl-2-oxo-1-[3-(trifluoromethyl)phenyl]-1,2,3,4-tetrahydropyrimidin-4-yl}benzonitrile

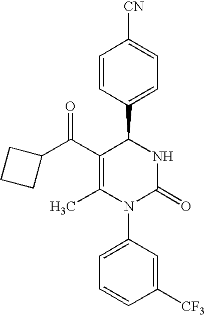

The racemic compound is prepared as described in WO 2005/082864 (Example 20). The racemate is separated into the enantiomers by preparative HPLC on a chiral phase (method 19).

HPLC (method 19): Rₜ=2.68 min

¹H-NMR (400 MHz, DMSO-d₆): δ=1.67 (m, 1H), 1.82 (m, 2H), 1.94 (s, 3H), 1.96-2.19 (m, 3H), 3.49 (dt, 1H), 5.37 (d, 1H), 7.53 (d, 1H), 7.61 (d, 2H), 7.68 (m, 2H), 7.78 (d, 1H), 7.89 (d, 2H), 8.42 (d, 1H).

### Example 4

### tert-Butyl[(6R)-6-(4-cyanophenyl)-5-(cyclobutylcarbonyl)-4-methyl-2-oxo-3-[3-(trifluoromethyl)-phenyl]-3,6-dihydropyrimidin-1(2H)-yl]acetate

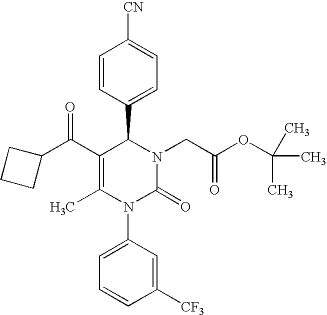

170 mg (0.387 mmol) of 4-{(4R)-5-(cyclobutylcarbonyl)-6-methyl-2-oxo-1-[3-(trifluoromethyl)-phenyl]-1,2,3,4-tetrahydropyrimidin-4-yl}benzonitrile are dissolved in 3 ml of DMF, and 91 mg (0.464 mmol) of tert-butyl bromoacetate and 96 mg (0.696 mmol) of potassium carbonate are added. The mixture is stirred at room temperature overnight. Then a further 91 mg (0.464 mmol) of tert-butyl bromoacetate and 50 mg (0.362 mmol) of potassium carbonate are added, and the suspension is stirred at 50° C. for a period of 6 hours. For working up, the mixture is added to 30 ml of water. The aqueous phase is extracted several times with ethyl acetate, the organic phases are combined, and the solvent is stripped off in a rotary evaporator. The residue is taken up in methanol and purified by preparative HPLC (method 14). 196 mg (92% of theory) of the target compound are obtained.

LC-MS (method 10): Rₜ=3.06 min; MS (ESIpos): m/z (%)=554.2 (18) [M+H]⁺.

### Example 5

### tert-Butyl[(6R)-6-(4-cyanophenyl)-5-(ethoxycarbonyl)-4-methyl-2-oxo-3-[3-(trifluoromethyl)-phenyl]-3,6-dihydropyrimidin-1(2H)-yl]acetate

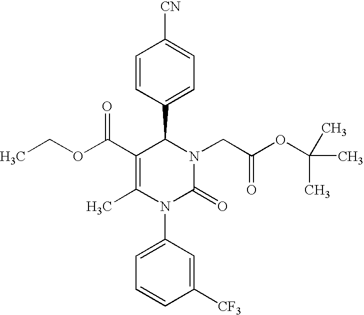

Under an argon protective gas atmosphere, ethyl(4R)-4-(4-cyanophenyl)-6-methyl-2-oxo-1-[3-(trifluoromethyl)phenyl]-1,2,3,4-tetrahydropyrimidine-5-carboxylate (10.74 g, 25 mmol) and solid potassium carbonate (5.53 g, 40 mmol) are introduced into dimethylformamide (100 ml). While stirring, tert-butyl bromoacetate (7.8 g, 40 mmol) is added dropwise. The reaction mixture is stirred at room temperature for 20 h and then reacted at 60° C. for a further 3 h. The reaction mixture is concentrated in vacuo, and the residue is taken up in ethyl acetate (200 ml). The organic phase is then washed with water (2×50 ml each time) and then with saturated aqueous sodium chloride solution (50 ml), dried over sodium sulfate, filtered and concentrated. The crude product is flash-chromatographed on silica gel (eluent: gradient of cyclohexane→cyclohexane/ethyl acetate 6:4). 12.5 g (91% of theory) of the title compound are obtained as a solid.

LC-MS (method 8): Rₜ=2.94 min; MS (ESIpos): m/z (%)=488.1 (100) [M+H—C₄H₈]⁺; MS (ESIneg): m/z (%)=542.2 (100) [M−H]⁻

¹H-NMR (400 MHz, DMSO-d₆): δ=1.05 (t, 3H), 1.25 (s, 9H), 2.05 (s, 3H), 3.85 (d, 1H), 4.0 (m, 3H), 5.55 (s, 1H), 7.60-7.90 (m, 8H).

### Example 6

### 2,3-Dihydroxypropyl(4R)-4-(4-cyanophenyl)-6-methyl-2-oxo-1-[3-(trifluoromethyl)phenyl]-1,2,3,4-tetrahydropyrimidine-5-carboxylate

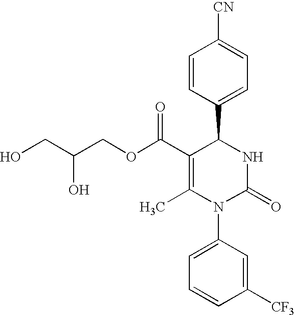

200 mg (0.453 mmol) of allyl(4R)-4-(4-cyanophenyl)-6-methyl-2-oxo-1-[3-(trifluoromethyl)-phenyl]-1,2,3,4-tetrahydropyrimidine-5-carboxylate (preparation according to WO 2005/082863, Example 6A) are dissolved in 20 ml of acetone/water (3:1). The solution is cooled to 0° C., and a solution of 72 mg (0.453 mmol) of potassium permanganate in 10 ml of acetone/water (3:1) is added. After stirring at room temperature for 90 minutes, saturated sodium thiosulfate solution is added, and the mixture is stirred for a further 30 minutes. The mixture is worked up by filtration with suction through a kieselguhr column which is eluted with a little methanol. After the organic phase has been separated off, the aqueous phase is back-extracted twice with ethyl acetate. The combined organic phases are concentrated, and the residue is purified by preparative HPLC (method 14). 119 mg (54% of theory) of the title compound are obtained.

LC-MS (method 8): Rₜ=2.06 min; MS (ESIpos): m/z (%)=476.1 (100) [M+H]⁺

¹H-NMR (400 MHz, DMSO-d₆): δ=2.05 (s, 3H), 3.19-3.31 (m, 2H), 3.60 (m, 1H), 3.89 (ddd, 1H), 4.05 (ddd, 1H), 4.61 (q, 1H), 4.92 (dd, 1H), 5.44 (m, 1H), 7.57 (br. s, 1H), 7.63 (m, 2H), 7.70 (m, 2H), 7.79 (d, 1H), 7.87 (d, 2H), 8.41 (m, 1H).

### Example 7

### 4-{(4R)-5-Acetyl-1-[4-fluoro-3-(trifluoromethyl)phenyl]-6-methyl-2-oxo-1,2,3,4-tetrahydropyrimidin-4-yl}benzonitrile

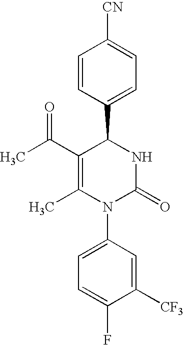

465 mg (4.646 mmol) of 2,4-pentanedione, 609 mg (4.646 mmol) of 4-formylbenzonitrile and 1200 mg (4.646 mmol) of N-[4-fluoro-3-(trifluoromethyl)phenyl]urea are dissolved in 12 ml of THF, and 3000 mg of ethyl polyphosphate (PPE) are added. The mixture is stirred under reflux for 6 hours and then concentrated in a rotary evaporator. The residue is purified firstly by flash chromatography on silica gel (eluent: cyclohexane/ethyl acetate 5:1→2:1→1:1) and then by preparative HPLC (method 14). The resulting racemate is fractionated by preparative HPLC on a chiral phase (method 17). 303 mg (16% of theory) of the title compound are obtained in this way.

HPLC (method 17): Rₜ=4.70 min

¹H-NMR (400 MHz, DMSO-d₆): δ=2.02 (s, 3H), 2.20 (s, 3H), 5.47 (d, 1H), 7.61 (m, 4H), 7.74 (br. s, 1H), 7.88 (d, 2H), 8.48 (d, 1H).

### Example 8

### [(6R)-6-(4-Cyanophenyl)-5-(cyclobutylcarbonyl)-4-methyl-2-oxo-3-[3-(trifluoromethyl)phenyl]-3,6-dihydropyrimidin-1(2H)-yl]acetic acid

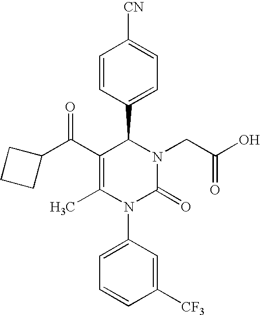

190 mg (0.343 mmol) of tert-butyl[(6R)-6-(4-cyanophenyl)-5-(cyclobutylcarbonyl)-4-methyl-2-oxo-3-[3-(trifluoromethyl)phenyl]-3,6-dihydropyrimidin-1(2H)-yl]acetate are dissolved in 3 ml of dichloromethane, 3 ml of trifluoroacetic acid are added, and the mixture is stirred at room temperature for two hours. The reaction mixture is concentrated and the residue is purified by preparative HPLC (method 14). 160 mg (94% of theory) of the title compound are obtained.

LC-MS (method 10): Rₜ=2.62 min; MS (ESIpos): m/z (%)=498.1 (100) [M+H]⁺

¹H-NMR (400 MHz, DMSO-d₆): δ=1.66 (m, 1H), 1.83 (m, 2H), 1.92 (s, 3H), 2.02 (m, 2H), 2.16 (m, 1H), 3.53 (dt, 1H), 3.84 (d, 1H), 4.18 (d, 1H), 5.60 (s, 1H), 7.59 (d, 1H), 7.65-7.74 (m, 4H), 7.81 (d, 1H), 7.88 (d, 2H), 12.70 (s, 1H).

### Example 9

### 5-{(4R)-5-Acetyl-6-methyl-2-oxo-1-[3-(trifluoromethyl)phenyl]-1,2,3,4-tetrahydropyrimidin-4-yl}-pyridine-2-carbonitrile

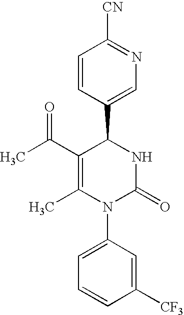

The title compound is prepared as described in WO 2004/024700 (Example 74).

### Example 10

### 2-Hydroxyethyl(4R)-4-(4-cyanophenyl)-6-methyl-2-oxo-1-[3-(trifluoromethyl)phenyl]-1,2,3,4-tetrahydropyrimidine-5-carboxylate

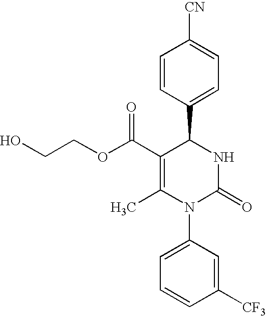

The title compound is prepared as described in WO 2004/024700 (Example 72).

### Example 11

### 2-(Dimethylamino)ethyl(4R)-4-(4-cyanophenyl)-6-methyl-2-oxo-1-[3-(trifluoromethyl)phenyl]-1,2,3,4-tetrahydropyrimidine-5-carboxylate trifluoroacetate

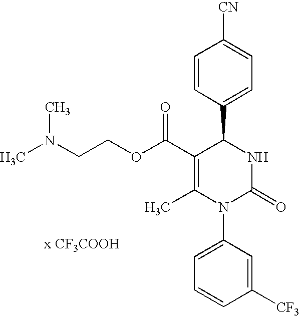

Under an argon protective gas atmosphere, 4-{(4R)-5-(1H-Imidazol-1-ylcarbonyl)-6-methyl-2-oxo-1-[3-(trifluoromethyl)phenyl]-1,2,3,4-tetrahydropyrimidin-4-yl}benzonitrile (Example 3A; 2.26 g, 5 mmol) is dissolved in N,N-dimethylethanolamine (20 ml) and then stirred at 100° C. for 3 h. The mixture is concentrated in vacuo, and the residue is taken up in ethyl acetate (150 ml) and then washed with water (50 ml) and saturated sodium chloride solution (50 ml). The organic phase is dried over sodium sulfate, filtered and concentrated. The residue is purified by preparative HPLC (method 22). 2.38 g (77% of theory) of the title compound are obtained.

LC-MS (method 10): Rₜ=1.2 min; MS (ESIpos): m/z (%)=473.5 (100) [M+H]⁺

¹H-NMR (400 MHz, DMSO-d₆): δ=2.05 (s, 3H), 2.10 (br. s, 6H), 2.40 (m, 2H), 4.10 (m, 2H), 5.40 (d, 1H), 7.55-7.70 (m, 5H), 7.80 (d, 1H), 7.90 (d, 2H), 8.40 (d, 1H).

### Example 12

### 4-{(4R)-5-Acetyl-1-[3-fluoro-5-(trifluoromethyl)phenyl]-6-methyl-2-oxo-1,2,3,4-tetrahydropyrimidin-4-yl}benzonitrile

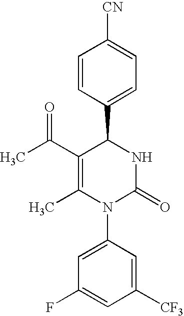

Under an argon protective gas atmosphere, 2,4-pentanedione (90.1 mg, 0.9 mmol) is introduced into THF (10 ml). 4-Cyanobenzaldehyde (118 mg, 0.9 mmol), N-[3-fluoro-5-(trifluoromethyl)phenyl]urea (200 mg, 0.9 mmol) and ethyl polyphosphate (551 mg) are successively added. The mixture is stirred under reflux until a TLC check indicates complete conversion (about 4 h). The mixture is concentrated in vacuo, and the residue is directly chromatographed (biotage medium pressure system, silica gel column, eluent: cyclohexane/ethyl acetate 5:1→3:1→2:1→1:1). 188 mg (50% of theory) of the racemic title compound are obtained. The racemate is subsequently separated into the enantiomers by chromatography (method 16). 28 mg of the enantiomerpure title compound (>99.5% ee) are obtained from 65 mg of the racemate.

HPLC (method 16): Rₜ=23.27 min

LC-MS (method 7): Rₜ=2.2 min; MS (ESIpos): m/z (%)=418.1 (100) [M+H]⁺

¹H-NMR (400 MHz, DMSO-d₆): δ=2.00 (s, 3H), 2.20 (s, 3H), 5.44 (d, 1H), 7.55 (m, 4H), 7.75 (br. s, 1H), 7.85 (d, 2H), 8.45 (d, 1H).

### Example 13

### [(6R)-6-(4-Cyanophenyl)-5-(ethoxycarbonyl)-4-methyl-2-oxo-3-[3-(trifluoromethyl)phenyl]-3,6-dihydropyrimidin-1(2H)-yl]acetic acid

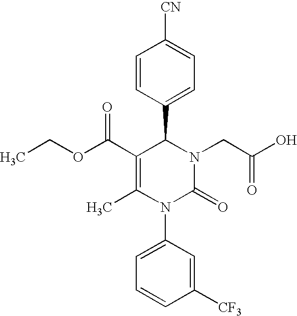

tert-Butyl[(6R)-6-(4-cyanophenyl)-5-(ethoxycarbonyl)-4-methyl-2-oxo-3-[3-(trifluoromethyl)phenyl]-3,6-dihydropyrimidin-1(2H)-yl]acetate (165 mg, 0.30 mmol) is introduced under an argon protective gas atmosphere into dichloromethane (1.5 ml), trifluoroacetic acid (1.5 ml) is added, and the mixture is stirred at room temperature for 4 h. The reaction mixture is concentrated in vacuo, and the residue is purified by preparative HPLC (method 13). 132 mg (89% of theory) of the title compound are obtained.

HPLC (method 2): Rₜ=3.0 min

LC-MS (method 7): Rₜ=2.3 min; MS (ESIpos): m/z (%)=488.1 (100) [M+H]⁺

¹H-NMR (400 MHz, DMSO-d₆): δ=1.10 (t, 3H), 2.05 (s, 3H), 3.75 (d, 1H), 4.05 (q, 2H), 4.10 (d, 1H), 5.60 (s, 1H), 7.60-7.75 (m, 5H), 7.80 (d, 1H), 7.90 (d, 2H), 12.70 (s, 1H).

### Example 14

### [(6R)-5-{[2-(Carboxymethoxy)ethoxy]carbonyl}-6-(4-cyanophenyl)-4-methyl-2-oxo-3-[3-(trifluoromethyl)phenyl]-3,6-dihydropyrimidin-1(2H)-yl]acetic acid

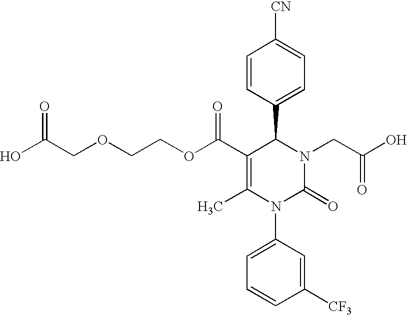

The title compound is prepared as described in WO 2005/082864 (Example 5).

### Example 15

### tert-Butyl[(6R)-6-(4-cyanophenyl)-5-isobutyryl-4-methyl-2-oxo-3-[3-(trifluoromethyl)phenyl]-3,6-dihydropyrimidin-1(2H)-yl]acetate

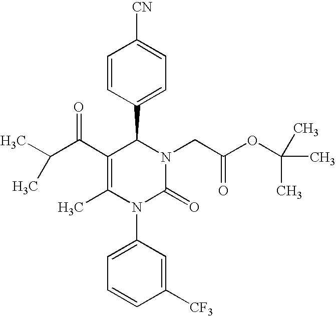

Under an argon protective gas atmosphere, sodium hydride (117 g, 29.2 mmol) is introduced into THF (50 ml). A solution of the compound from Example 2 (5.0 g, 11.7 mmol) in THF (150 ml) is slowly added, and the reaction mixture is stirred while foaming at RT for 1 h. tert-Butyl bromoacetate (3.4 g, 17.5 mmol) is then slowly added. After 2 h, a thin-layer chromatogram shows complete conversion. Water (300 ml) is cautiously added to the mixture. The aqueous phase is saturated solid sodium chloride and extracted three times with ethyl acetate. The combined organic phases are dried over sodium sulfate, filtered and concentrated in vacuo. The residue is absorbed on silica gel and purified by MPLC (200 g of silica gel, eluent: cyclohexane/ethyl acetate 4:1). 5 g of the title compound are obtained with a purity of 79%, and 4.4 g with a purity of 70%, which can be further purified by preparative HPLC.

LC-MS (method 7): Rₜ=2.8 min; MS (ESIpos): m/z (%)=486 (100) [M−C₄H₈+H]⁺;

MS (ESIneg): m/z (%)=540 (100) [M−H]⁻.

For the further analytical data, see those reported for the racemic compound (WO 2005/082863, Example 34A).

### Example 16

### [(6R)-6-(4-Cyanophenyl)-5-isobutyryl-4-methyl-2-oxo-3-[3-(trifluoromethyl)phenyl]-3,6-dihydropyrimidin-1(2H)-yl]acetic acid

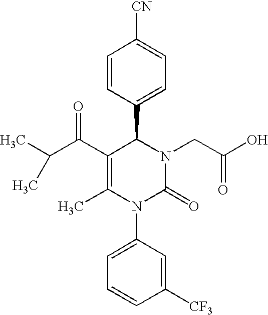

tert-Butyl[(6R)-6-(4-cyanophenyl)-5-isobutyryl-4-methyl-2-oxo-3-[3-(trifluoromethyl)phenyl]-3,6-dihydropyrimidin-1(2H)-yl]acetate (4.43 g, 8.2 mmol) is introduced under an argon protective gas atmosphere into dichloromethane (50 ml), trifluoroacetic acid (18.7 g, 164 mmol) is added, and the mixture is stirred at room temperature for about 4 h (until a TLC check indicates complete conversion). The reaction mixture is concentrated in vacuo, and the residue is purified by chromatography (Biotage chromatography system, silica gel, eluent: dichloromethane→dichloromethane/methanol 100:1→50:1). 1.44 g (34% of theory) of the title compound are obtained.

LC-MS (method 7): Rₜ=2.3 min; MS (ESIpos): m/z (%)=486.1 (100) [M+H]⁺;

MS (ESIneg): m/z (%)=484.2 (80) [M−H]⁻, 969.5 (100)

¹H-NMR (400 MHz, DMSO-d₆): δ=0.60 (d, 3H), 0.70 (d, 3H), 1.60 (s, 3H), 2.70 (m, 1H), 3.60 (d, 1H), 4.00 (d, 1H), 5.50 (s, 1H), 7.35-7.50 (m, 5H), 7.60 (d, 1H), 7.65 (d, 2H), 12.50 (br. s, 1H).

### Example 17

### 3-{[(6R)-5-Acetyl-6-(4-cyanophenyl)-4-methyl-2-oxo-3-[3-(trifluoromethyl)phenyl]-3,6-dihydropyrimidin-1(2H)-yl]methyl}benzoic acid

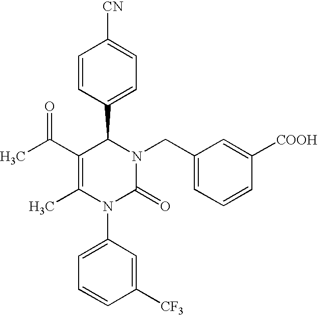

The title compound is prepared as described in WO 2005/082863 (Example 21).

### Example 18

### 4-{[(6R)-5-Acetyl-6-(4-cyanophenyl)-4-methyl-2-oxo-3-[3-(trifluoromethyl)phenyl]-3,6-dihydropyrimidin-1(2H)-yl]methyl}benzoic acid

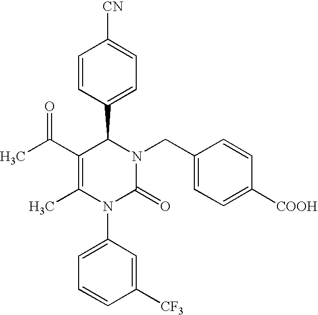

The title compound is prepared as described in WO 2005/082863 (Example 22).

### Example 19

### tert-Butyl[(6R)-5-acetyl-6-(4-cyanophenyl)-4-methyl-2-oxo-3-[3-(trifluoromethyl)phenyl]-3,6-dihydropyrimidin-1(2H)-yl]acetate

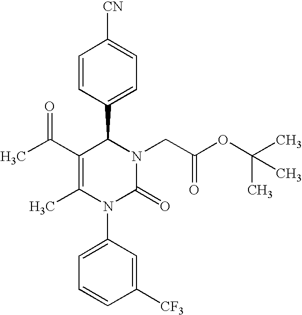

The reaction is carried out under argon. Solid sodium hydride (3.76 g, 93.9 mmol) is added in portions to a solution of {4R}-4-{5-acetyl-6-methyl-2-oxo-1-[3-(trifluoromethyl)phenyl]-1,2,3,4-tetrahydro-4-pyrimidinyl}benzonitrile (15.0 g, 37.6 mmol; WO 2005/082864, Example 18A) in THF (450 ml). The mixture is stirred at RT for 1 h and then tert-butyl bromoacetate (11 g, 56.3 mmol) is slowly added, and the mixture is again stirred at RT for 1 h. A TLC check shows complete conversion. Water (500 ml) is cautiously added to the mixture. Saturated aqueous sodium chloride solution is added, and the mixture is then extracted three times with ethyl acetate. The combined organic phases are dried over sodium sulfate, filtered and concentrated in vacuo. The crude product is purified by MPLC on silica gel (eluent: cyclohexane/methylene chloride 1:1). A colorless solid is obtained as product (9.94 g, 51% of theory) which can be employed in the next reaction without further purification steps.

¹H-NMR (400 MHz, DMSO-d₆): δ=1.25 (s, 9H), 2.00 (s, 3H), 2.20 (s, 3H), 3.95 (d, 1H), 4.15 (d, 1H), 5.70 (s, 1H), 7.60-7.90 (m, 8H).

### Example 20

### [(6R)-5-Acetyl-6-(4-cyanophenyl)-4-methyl-2-oxo-3-[3-(trifluoromethyl)phenyl]-3,6-dihydropyrimidin-1(2H)-yl]acetic acid

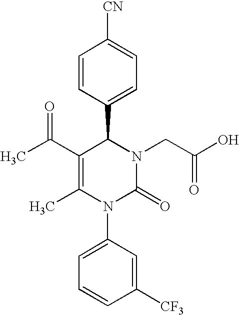

The reaction is carried out under argon. Trifluoroacetic acid (36.5 ml, 473.2 mmol) is added to a solution of the compound from Example 19 (12.2 g, 23.7 mmol) in dichloromethane (160 ml). The reaction mixture is stirred at RT overnight. A TLC check then shows complete conversion. The mixture is subsequently concentrated in vacuo, and the residue is purified by flash chromatography on silica gel (eluent: methylene chloride→methylene chloride/methanol 100:1→methylene chloride/methanol 50: 1). A solid is obtained as product (7.55 g, 70% of theory).

¹H-NMR (400 MHz, DMSO-d₆): δ=1.95 (s, 3H), 2.25 (s, 3H), 2.95 (d, 1H), 4.15 (d, 1H), 5.80 (s, 1H), 7.55-7.90 (m, 8H).

### Example 21

### 2-[(6R)-5-Acetyl-6-(4-cyanophenyl)-4-methyl-2-oxo-3-[3-(trifluoromethyl)phenyl]-3,6-dihydropyrimidine-1(2H)-yl]acetamide

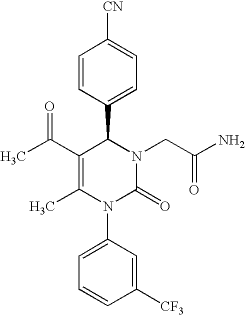

The reaction is carried out under argon. [(6R)-5-Acetyl-6-(4-cyanophenyl)-4-methyl-2-oxo-3-[3-(trifluoromethyl)phenyl]-3,6-dihydropyrimidin-1(2H)-yl]acetic acid from Example 20 (12.0 g, 26.2 mmol) is introduced into DMF (150 ml) and, at 0° C., HATU (19.95 g, 52.5 mmol) is added, and the mixture is stirred for 20 min. Then ammonium chloride (7.0 g, 131.2 mmol) and N,N-diisopropylethylamine (23.7 g, 183.6 mmol) are added, and the mixture is stirred at RT for 90 min. HPLC or TLC check then show complete conversion. The reaction mixture is concentrated in vacuo. The residue is then taken up in ethyl acetate (400 ml) and washed successively with aqueous sodium bicarbonate solution (70 ml), 10% strength citric acid solution (3×70 ml each time), sodium bicarbonate solution (70 ml) and saturated sodium chloride solution (70 ml). The organic phase is dried over sodium sulfate, filtered and concentrated in vacuo, and the residue is dried under high vacuum. The resulting crude product is purified by preparative HPLC. For this purpose, the crude product is dissolved in a mixture of acetonitrile (80 ml), methanol (60 ml), water (20 ml) and trifluoroacetic acid (1 ml). Kromasil 100 C18 5 μm is used as solid phase (column dimensions: 250 mm×20 mm). Isocratic elution is carried out with 0.2% strength TFA/acetonitrile 1:1. A colorless solid is obtained as product (9.1 g, 76% of theory) in this way.

LC-MS (method 8): Rₜ=2.2 min; MS (ESIpos): m/z (%)=457.0 (100) [M+H]⁺, 439.9 (60); MS (ESIneg): m/z (%)=455.0 (100) [M−H]⁻

¹H-NMR (400 MHz, DMSO-d₆): δ=1.95 (s, 3H), 2.25 (s, 3H), 3.40 (d, 1H), 4.15 (d, 1H), 5.65 (s, 1H), 7.15 (br. s, 1H), 7.45 (br. s, 1H), 7.60-7.90 (m, 8H).

### Example 22

### Ethyl(4R)-3-(2-amino-2-oxoethyl)-4-(4-cyanophenyl)-6-methyl-2-oxo-1-[3-(trifluoromethyl)-phenyl]-1,2,3,4-tetrahydropyrimidine-5-carboxylate

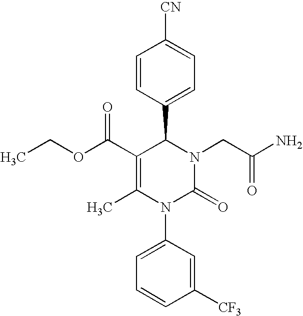

100 mg (0.205 mmol) of [(6R)-6-(4-cyanophenyl)-5-(ethoxycarbonyl)-4-methyl-2-oxo-3-[3-(trifluoromethyl)phenyl]-3,6-dihydropyrimidin-1(2H)-yl]acetic acid from Example 13 are dissolved in 7.5 ml of THF and cooled to −20° C. 52 mg (0.513 mmol) of N-methylmorpholine and 56 mg (0.513 mmol) of ethyl chloroformate are added to this solution. The mixture is stirred at −20° C. for 30 min and then a mixture of 0.2 ml of 35% strength aqueous ammonia solution in 1.5 ml of THF is added. The reaction mixture is then allowed slowly to reach room temperature and the contents of the flask are then put in 3 N hydrochloric acid. The aqueous phase is extracted with ethyl acetate and separated off. The organic phase is dried over sodium sulfate, filtered and concentrated. The residue is purified by preparative HPLC (method 23). 78 mg (78% of theory) of the title compound are obtained in this way.

MS (ESIpos): m/z (%)=487 (100) [M+H]⁺

HPLC (method 4): Rₜ=4.31 min

¹H-NMR (400 MHz, DMSO-d₆): δ=1.12 (t, 3H), 2.03 (s, 3H), 3.31 (d, 1H), 4.05 (dq, 2H), 4.12 (d, 1H), 5.51 (s, 1H), 7.16 (s, 1H), 7.44 (s, 1H), 7.63-7.66 (m, 3H), 7.73 (t, 1H), 7.78 (s, 1H), 7.82 (d, 1H), 7.89 (d, 2H).

### Example 23

### (4R)-4-(4-Cyanophenyl)-6-methyl-2-oxo-1-[3-(trifluoromethyl)phenyl]-1,2,3,4-tetrahydropyrimidine-5-carbonitrile

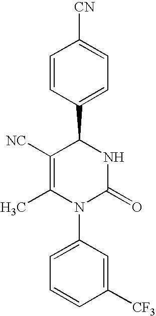

The title compound is prepared as described in WO 2005/082863 (Example 15A).

### Example 24

### tert-Butyl[(6R)-5-cyano-6-(4-cyanophenyl)-4-methyl-2-oxo-3-[3-(trifluoromethyl)phenyl]-3,6-dihydropyrimidin-1(2H)-yl]acetate

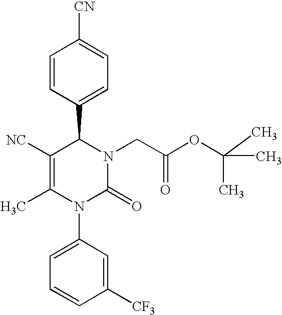

5900 mg (15.431 mmol) of (4R)-4-(4-cyanophenyl)-6-methyl-2-oxo-1-[3-(trifluoromethyl)phenyl]-1,2,3,4-tetrahydropyrimidine-5-carbonitrile are dissolved in 150 ml of DMF, and 7677 mg (55.551 mmol) of potassium carbonate and 7224 mg (37.034 mmol) of tert-butyl bromoacetate are added. The reaction mixture is then stirred at 60° C. overnight. The solvent is subsequently stripped off in a rotary evaporator, and the residue is taken up in a mixture of ethyl acetate and water. The organic phase is separated off, dried over sodium sulfate, filtered and concentrated. The residue is absorbed on silica gel and purified by MPLC on silica gel (eluent: cyclohexane/ethyl acetate 1:2). 5570 mg (73% of theory) of the title compound are obtained in this way.

LC-MS (method 6): Rₜ=2.65 min; MS (ESIneg): m/z (%)=495.2 (100) [M−H]⁻

¹H-NMR (400 MHz, DMSO-d₆): δ=1.29 (s, 9H), 1.86 (s, 3H), 3.71 (d, 1H), 3.96 (d, 1H), 5.49 (s, 1H), 7.70-7.75 (m, 2H), 7.78 (d, 2H), 7.84 (d, 1H), 7.87 (s, 1H), 7.96 (d, 2H).

### Example 25

### [(6R)-5-Cyano-6-(4-cyanophenyl)-4-methyl-2-oxo-3-[3-(trifluoromethyl)phenyl]-3,6-dihydropyrimidin-1(2H)-yl]acetic acid

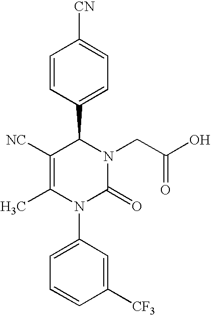

5840 mg (11.762 mmol) of tert-butyl[(6R)-5-cyano-6-(4-cyanophenyl)-4-methyl-2-oxo-3-[3-(trifluoromethyl)phenyl]-3,6-dihydropyrimidin-1(2H)-yl]acetate are dissolved in 40 ml of dichloromethane, and 13412 mg (117.625 mmol) of trifluoroacetic acid are added. The mixture is stirred at 50° C. for 5 h. The volatile constituents are then stripped off in a rotary evaporator. The residue is purified by MPLC on silica gel (eluent: dichloromethane/methanol 10: 1). 3120 mg (58% of theory) of the title compound are obtained in this way.

LC-MS (method 10): Rₜ=2.47 min; MS (ESIpos): m/z (%)=441.1 (100) [M+H]⁺.

### Example 26

### 2-[(6R)-5-Cyano-6-(4-cyanophenyl)-4-methyl-2-oxo-3-[3-(trifluoromethyl)phenyl]-3,6-dihydropyrimidin-1(2H)-yl]acetamide

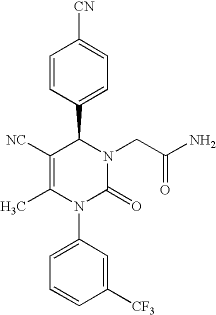

100 mg (0.227 mmol) of [(6R)-5-cyano-6-(4-cyanophenyl)-4-methyl-2-oxo-3-[3-(trifluoromethyl)-phenyl]-3,6-dihydropyrimidin-1(2H)-yl]acetic acid are dissolved in 5 ml of THF and cooled to −20° C. 57 mg (0.568 mmol) of N-methylmorpholine and 62 mg (0.568 mmol) of ethyl chloroformate are added to this solution. The mixture is stirred at −20° C. for 30 min and then a mixture of 0.15 ml of 35% strength aqueous ammonia solution in 1.5 ml of THF is added. The reaction mixture is then allowed slowly to reach room temperature, and the contents of the flask are then put into 3N hydrochloric acid. The aqueous phase is extracted with ethyl acetate and separated off. The organic phase is dried over sodium sulfate, filtered and concentrated. The residue is purified by preparative HPLC (method 23). 44 mg (44% of theory) of the title compound are obtained in this way.

MS (DCI): m/z (%)=457 (100) [M+NH₄]⁺, 440 (6) [M+H]⁺

HPLC (method 3): Rₜ=4.19 min

¹H-NMR (400 MHz, DMSO-d₆): δ=1.82 (s, 3H), 3.20 (d, 1H), 4.10 (d, 1H), 5.45 (s, 1H), 7.12 (s, 1H), 7.39 (s, 1H), 7.72-7.76 (m, 4H), 7.83 (d, 1H), 7.87 (s, 1H), 7.89 (d, 2H).

### Example 27

### tert-Butyl[(6R)-6-(4-cyanophenyl)-5-[(2-hydroxyethoxy)carbonyl]-4-methyl-2-oxo-3-[3-(trifluoromethyl)phenyl]-3,6-dihydropyrimidin-1(2H)-yl]acetate

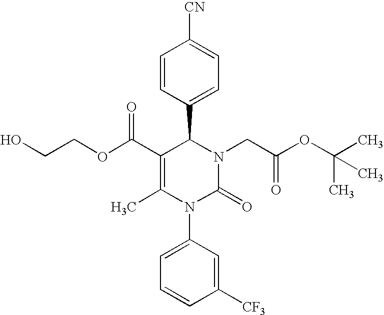

(4R)-3-(2-tert-Butoxy-2-oxoethyl)-4-(4-cyanophenyl)-6-methyl-2-oxo-1-[3-(trifluoromethyl)phenyl]-1,2,3,4-tetrahydropyrimidine-5-carboxylic acid (Example 4A; 515 mg, 1 mmol), 2-bromoethanol (521 mg, 4 mmol) and N,N-diisopropylethylamine (258 mg, 2 mmol) are stirred in DMF (15 ml) at 70° C. for 24 h. The reaction mixture is then concentrated in vacuo, and the residue is taken up in ethyl acetate. The organic phase is washed with water and then with concentrated aqueous sodium chloride solution. It is then dried over sodium sulfate, filtered and concentrated. The crude product is purified by flash chromatography on silica gel (eluent: cyclohexane→cyclohexane/ethyl acetate 1:2). A colorless solid is obtained as product (472 mg, 84% of theory).

LC-MS (method 8): Rₜ=2.6 min; MS (ESIpos): m/z (%)=504.1 (100), 560 (20) [M+H]⁺; MS (ESIneg): m/z (%)=558.4 (100) [M−H]⁻.

### Example 28

### [(6R)-6-(4-Cyanophenyl)-5-[(2-hydroxyethoxy)carbonyl]-4-methyl-2-oxo-3-[3-(trifluoromethyl)-phenyl]-3,6-dihydropyrimidin-1(2H)-yl]acetic acid

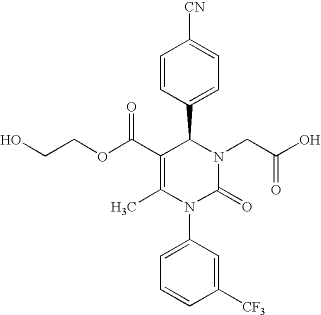

tert-Butyl[(6R)-6-(4-cyanophenyl)-5-[(2-hydroxyethoxy)carbonyl]-4-methyl-2-oxo-3-[3-trifluoromethyl)phenyl]-3,6-dihydropyrimidin-1(2H)-yl]acetate (Example 27; 235 mg, 0.42 mmol) is introduced into dichloromethane (2 ml). Trifluoroacetic acid (2 ml) is then added, and the mixture is stirred at RT for 24 h. The reaction mixture is then concentrated in vacuo and the crude product is purified by preparative HPLC (column: Kromasil 5 μm; eluent: acetonitrile/water+0.1% TFA 10:90→90:10). The title compound is obtained as a solid (115 mg, 54% of theory).

LC-MS (method 7): Rₜ=1.9 min; MS (ESIpos): m/z (%)=504.1 (100) [M+H]⁺; MS (ESIneg): m/z (%)=502.1 (70) [M−H]⁻.

### Example 29

### 2-Hydroxyethyl(4R)-3-(2-amino-2-oxoethyl)-4-(4-cyanophenyl)-6-methyl-2-oxo-1-[3-trifluoromethyl)phenyl]-1,2,3,4-tetrahydropyrimidine-5-carboxylate

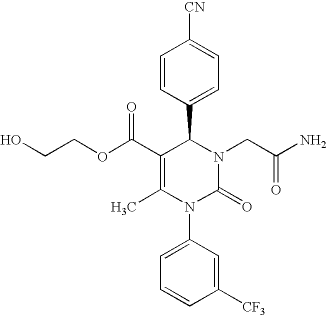

The reaction is carried out under argon. [(6R)-6-(4-Cyanophenyl)-5-[(2-hydroxyethoxy)carbonyl]-4-methyl-2-oxo-3-[3-(trifluoromethyl)phenyl]-3,6-dihydropyrimidin-1(2H)-yl]acetic acid (Example 28; 40 mg, 79 μmol) is introduced into DMF (2 ml) and, at 0° C., HATU (151 mg, 0.4 mmol) is added, and the mixture is stirred for 20 min. Then ammonium chloride (21 mg, 0.4 mmol) and N,N-diisopropylethylamine (103 mg, 0.8 mmol) are added, and the reaction mixture is stirred at RT for 16 h. The resulting crude mixture is purified directly by preparative HPLC (column: Gromsil C18 10 μm; eluent: acetonitrile/water+0.1% TFA 10:90→90:10). The title compound is obtained as a colorless solid (16 mg, 40% of theory).

LC-MS (method 24): Rₜ=2.9 min; MS (ESIpos): m/z (%)=503.2 (60) [M+H]⁺, 441.2 (100); MS (ESIneg): m/z (%)=501.2 (100) [M−H]⁻

¹H-NMR (400 MHz, DMSO-d₆): δ=2.00 (s, 3H), 3.30 (d, 1H), 3.50 (t, 2H), 4.05 (m, 2H), 4.15 (d, 1H), 5.55 (s, 1H), 7.15 (br. s, 1H), 7.50 (br. s, 1H), 7.60-7.90 (m, 8H).

### Example 30

### Ethyl(4R)-4-(4-cyanophenyl)-3-[2-(dimethylamino)-2-oxoethyl]-6-methyl-2-oxo-1-[3-(trifluoromethyl)phenyl]-1,2,3,4-tetrahydropyrimidine-5-carboxylate

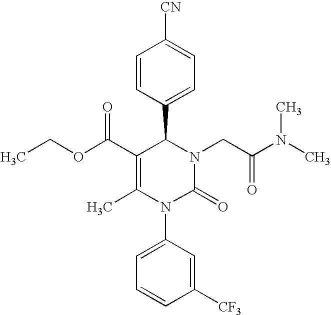

975 mg (2.00 mmol) of [(6R)-6-(4-cyanophenyl)-5-(ethoxycarbonyl)-4-methyl-2-oxo-3-[3-(trifluoromethyl)phenyl]-3,6-dihydropyrimidin-1(2H)-yl]acetic acid (Example 13) are dissolved in 4 ml of DMF, and 2.2 ml (4.401 mmol) of a 2 M solution of dimethylamine in THF are added. Then 449 mg (4.00 mmol) of 4-N,N-dimethylaminopyridine, 595 mg (4.401 mmol) of 1-hydroxy-1H-benzotriazole hydrate and 844 mg (4.401 mmol) of 1-(3-dimethylaminopropyl)-3-ethylcarbodiimide hydrochloride are added. The mixture is stirred at RT overnight. After the mixture has been concentrated in a rotary evaporator, the crude product is purified by preparative HPLC (method 23). 795 mg (77% of theory) of the title compound are obtained in this way.

LC-MS (method 25): Rₜ=3.67 min; MS (ESIpos): m/z (%)=515.4 (100) [M+H]⁺.

### B. ASSESSMENT OF PHARMACOLOGICAL ACTIVITY

The pharmacological effect of the compounds of the invention can be shown in the assays described below:

Abbreviations:

AMC 7-Amido-4-methylcoumarin

BNP brain natriuretic peptide

BSA bovine serum albumin

HEPES N-(2-Hydroxyethyl)piperazine-N′-2-ethanesulfonic acid

HNE humane neutrophil elastase

IC Inhibitory concentration

MeOSuc Methoxysuccinyl

NADP Nicotinamide adenine dinucleotide phosphate

v/v Volume to volume ratio (of a solution)

w/v Weight to volume ratio (of a solution)

B-1. In vitro HNE Inhibition Assay

The potency of the compounds of the invention is ascertained in an in vitro inhibition assay. The HNE-mediated amidolytic cleavage of a suitable peptide substrate leads in this connection to an increase in the fluorescent light. The signal intensity of the fluorescent light is directly proportional to the enzyme activity. The effective concentration of a test compound at which half the enzyme is inhibited (50% signal intensity of the fluorescent light) is indicated as IC₅₀.

Procedure:

Enzyme (80 pM HNE; from Serva, Heidelberg) and substrate (20 μM MeOSuc-Ala-Ala-Pro-Val-AMC; from Bachem, Weil am Rhein) are incubated in an assay volume of in total 50 μl of assay buffer (0.1 M HEPES pH 7.4, 0.5 M NaCl, 0.1% w/v BSA, 1% v/v DMSO) in a 384-well microtiter plate in the presence and absence of the test substance at 37° C. for 2 hours. The intensity of the fluorescent light from the assay mixtures is measured (Ex. 380 nm, Em. 460 nm). The IC₅₀ values are determined by plotting the intensity of the fluorescent light against the active substance concentration.

Representative IC₅₀ values for the compounds of the invention are shown in the following table:

TABLE
Inhibition of human neutrophil elastase (HNE)

| Exemplary embodiment No. | IC₅₀ [nM] |
|---|---|
| 3 | 14.8 |
| 7 | 5.2 |
| 8 | 4.3 |
| 9 | 20.0 |
| 10 | 4.6 |
| 13 | 2.1 |
| 14 | 0.9 |
| 16 | 2.9 |
| 17 | 3.9 |
| 18 | 2.9 |
| 21 | 5.5 |
| 26 | 5.4 |
| 29 | 1.4 |
| 30 | 0.4 |

B-2. Animal Model of Pulmonary Arterial Hypertension

The monocrotaline-induced pulmonary hypertension in rats is a widely used animal model of pulmonary arterial hypertension. The pyrrolizidine alkaloid monocrotaline is metabolized after subcutaneous injection to the toxic monocrotalinepyrrole in the liver and leads within a few days to endothelial damage in the pulmonary circulation, followed by a remodeling of the small pulmonary arteries (media hypertrophy, de novo muscularization). A single subcutaneous injection is sufficient to induce pronounced pulmonary hypertension in rats within 4 weeks [Cowan et al., Nature Med. 6 698-702 (2000)].

Male Sprague-Dawley rats are used for the model. On day 0, the animals receive a subcutaneous injection of 60 mg/kg monocrotaline. Treatment of the animals begins no earlier than 14 days after the monocrotaline injection and extends over a period of at least 14 days. At the end of the study, the animals undergo hemodynamic investigations, and the arterial and central venous oxygen saturation are determined. For the hemodynamic measurement, the rats are initially anesthetized with pentobarbital (60 mg/kg). The animals are then tracheotomized and artificially ventilated (rate: 60 inspirations/min; inspiration to expiration ratio: 50:50; positive end-expiratory pressure: 1 cm H₂O; tidal volume: 10 ml/kg of body weight; FIO₂: 0.5). The anesthesia is maintained by isoflurane inhalation anesthesia. The systemic blood pressure is determined in the left carotid artery using a Millar microtip catheter. A polyethylene catheter is advanced through the right jugular vein into the right ventricle to determine the right ventricular pressure. The cardiac output is determined by thermodilution. Following the hemodynamics, the heart is removed and the ratio of right to left ventricle including septum is determined. In addition, plasma samples are obtained to determine biomarkers (for example proBNP) and plasma substance levels.

B-3. CYP Inhibition Assay

The ability of substances to be able to inhibit CYP1A2, CYP2C9, CYP2D6 and CYP3A4 in humans is investigated with pooled human liver microsomes as enzyme source in the presence of standard substrates (see below) which form CYP-specific metabolites. The inhibitory effects are investigated with six different concentrations of the test compounds (2.8, 5.6, 8.3, 16.7, 25 and 50 μM), compared with the extent of the CYP-specific metabolite formation of the standard substrates in the absence of the test compounds, and the corresponding IC₅₀ values are calculated. A standard inhibitor which specifically inhibits a single CYP isoform is always included in the incubation in order to make the results comparable between different series.

Procedure:

Incubation of phenacetin, diclofenac, tolbutamide, dextromethorphan or midazolam with human liver microsomes in the presence of in each case six different concentrations of a test compound (as potential inhibitor) is carried out on a work station (Tecan, Genesis, Crailsheim, Germany). Standard incubation mixtures comprise 1.3 mM NADP, 3.3 mM MgCl₂×6 H₂O, 3.3 mM glucose 6-phosphate, glucose 6-phosphate dehydrogenase (0.4 U/ml) and 100 mM phosphate buffer (pH 7.4) in a total volume of 200 μl. Test compounds are preferably dissolved in acetonitrile. 96-well plates are incubated with pooled human liver microsomes at 37° C. for a defined time. The reactions are stopped by adding 100 μl of acetonitrile in which a suitable internal standard is always present. Precipitated proteins are removed by centrifugation, and the supernatants are combined and analyzed by LC-MS/MS.

B-4. Hepatocyte Assay to Determine the Metabolic Stability

The metabolic stability of test compounds in the presence of hepatocytes is determined by incubating the compounds with low concentrations (preferably below 1 μM) and with low cell counts (preferably 1*10⁶ cells/ml) in order to ensure as far as possible linear kinetic conditions in the experiment. Seven samples of the incubation solution are taken in a fixed time pattern for the LD-MS analysis in order to determine the half-life (i.e. the degradation) of the compound. Various clearance parameters (CL) and Fₘₐₓ values are calculated from this half-life (see below).

The Cl and Fₘₐₓ values represent a measure of the phase 1 and phase 2 metabolism of the compound in the hepatocytes. In order to minimize the influence of the organic solvent on the enzymes in the incubation mixtures, this concentration is generally limited to 1% (acetonitrile) or 0.1% (DMSO).

A cell count for hepatocytes in the liver of 1.1*10⁸ cells/g of liver is used for calculation of all species and breeds. CL parameters calculated on the basis of half-lives extending beyond the incubation time (normally 90 minutes) can be regarded only as rough guidelines.

The calculated parameters and their meaning are:

| | |
|---|---|
| Fₘₐₓ well-stirred [%] | Maximum possible bioavailability after oral administration |
| Calculation: | (1 − CL_{blood} well-stirred/QH) * 100 |
| CL_{blood} well-stirred [L/(h*kg)] | calculated blood clearance (well stirred model) |
| Calculation: | (QH * CL′_{intrinsic})/(QH + CL′_{intrinsic}) |
| CL′_{intrinsic} [ml/(min*kg)] | maximum ability of the liver (of the hepatocytes) to metabolize |
| | a compound (on the assumption that the hepatic blood flow is |
| | not rate-limiting) |
| Calculation: | CL′_{intrinsic, apparent} * species-specific hepatocyte count [1.1 * 10⁸/g |
| | of liver] * species-specific liver weight [g/kg] |
| CL′_{intrinsic, apparent} [ml/(min*mg)] | normalizes the elimination constant by dividing it by the |
| | hepatocyte cell count x (x * 10⁶/ml) employed |
| Calculation: | kₑₗ [1/min]/(cell count [x * 10⁶]/incubation volumes [ml]) |

(QH = species-specific hepatic blood flow).

### C. EXEMPLARY EMBODIMENTS OF PHARMACEUTICAL COMPOSITIONS

The compounds of the invention can be converted into pharmaceutical preparations in the following ways:

Tablet:

Composition:

100 mg of the compound of the invention, 50 mg of lactose (monohydrate), 50 mg of corn starch (native), 10 mg of polyvinylpyrrolidone (PVP 25) (from BASF, Ludwigshafen, Germany) and 2 mg of magnesium stearate.

Tablet weight 212 mg, diameter 8 mm, radius of curvature 12 mm.

Production:

The mixture of compound of the invention, lactose and starch is granulated with a 5% strength solution (m/m) of the PVP in water. The granules are mixed with the magnesium stearate for 5 minutes after drying. This mixture is compressed with a conventional tablet press (see above for format of the tablet). A guideline compressive force for the compression is 15 kN.

Suspension Which can be Administered Orally:

Composition:

1000 mg of the compound of the invention, 1000 mg of ethanol (96%), 400 mg of Rhodigel® (xanthan gum from FMC, Pennsylvania, USA) and 99 g of water.

10 ml of oral suspension correspond to a single dose of 100 mg of the compound of the invention.

Production:

The Rhodigel is suspended in ethanol, and the compound of the invention is added to the suspension. The water is added while stirring. The mixture is stirred for about 6 h until the swelling of the Rhodigel is complete.

Solution Which can be Administered Orally:

Composition:

500 mg of the compound of the invention, 2.5 g of polysorbate and 97 g of polyethylene glycol 400. 20 g of oral solution correspond to a single dose of 100 mg of the compound according to the invention.

Production:

The compound of the invention is suspended in the mixture of polyethylene glycol and polysorbate with stirring. The stirring process is continued until the compound according to the invention has completely dissolved.

i.v. Solution:

The compound of the invention is dissolved in a concentration below the saturation solubility in a physiologically tolerated solvent (e.g. isotonic saline solution, 5% glucose solution and/or 30% PEG 400 solution). The solution is sterilized by filtration and used to fill sterile and pyrogen-free injection containers.

1. The use of a compound of the formula (I) 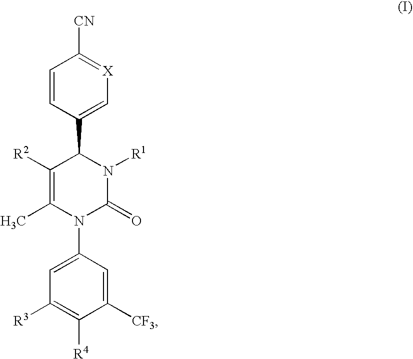
in which
X is CH or N,
R¹ is hydrogen, a group of the formula —(CH₂)ₙ—C(═O)—O—R^{1A} or —(CH₂)ₙ—C(═O)—NR^{1B}R^{1C} or a group of the formula 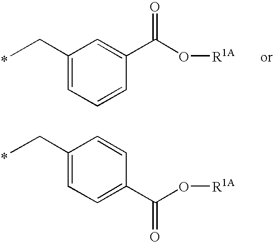
in which
* means the point of linkage to the N atom,
n is the number 1 or 2,
R^{1A} is hydrogen or (C₁-C₄)-alkyl,
and
R^{1B} and R^{1C} are independently of one another hydrogen or (C₁-C₄)-alkyl,
R² is cyano or a group of the formula —C(═O)—R^{2A} or —C(═O)—O—R^{2A}, in which
R^{2A} is (C₁-C₆)-alkyl or (C₃-C₆)-cycloalkyl each of which in turn may be substituted up to twice, identically or differently, by hydroxy, (C₁-C₄)-alkoxy, hydroxycarbonyl, amino, mono- and/or di-(C₁-C₄)-alkylamino, and in which in each case a CH₂ group can be replaced by an O atom,
and
R³ either is hydrogen
and
R⁴ is hydrogen, fluorine or chlorine,
or
R³ is fluorine or chlorine
and
R⁴ is hydrogen,
or of one of the salts, solvates and solvates of the salts thereof for the manufacture of a medicament for the treatment and/or prophylaxis of pulmonary arterial hypertension and other types of pulmonary hypertension.

2. The use as claimed in claim 1 of a compound of the formula (I) as claimed in claim 1 in which
X is CH or N,
R¹ is hydrogen, a group of the formula —CH₂—C(═O)—OH or —CH₂—C(═O)—NH₂ or a group of the formula 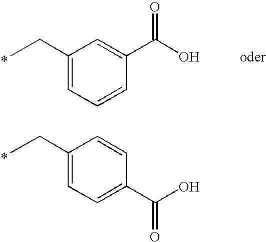
in which
* means the point of linkage to the N atom,
R² is cyano, acetyl, cyclobutylcarbonyl, methoxycarbonyl, ethoxycarbonyl or 2-hydroxyethoxycarbonyl,
R³ is hydrogen,
and
R⁴ is hydrogen or fluorine,
or of one of the salts, solvates and solvates of the salts thereof.

3. The use as claimed in claim 1 of a compound of formula (I) as claimed in claim 1 selected from the group of compounds consisting of 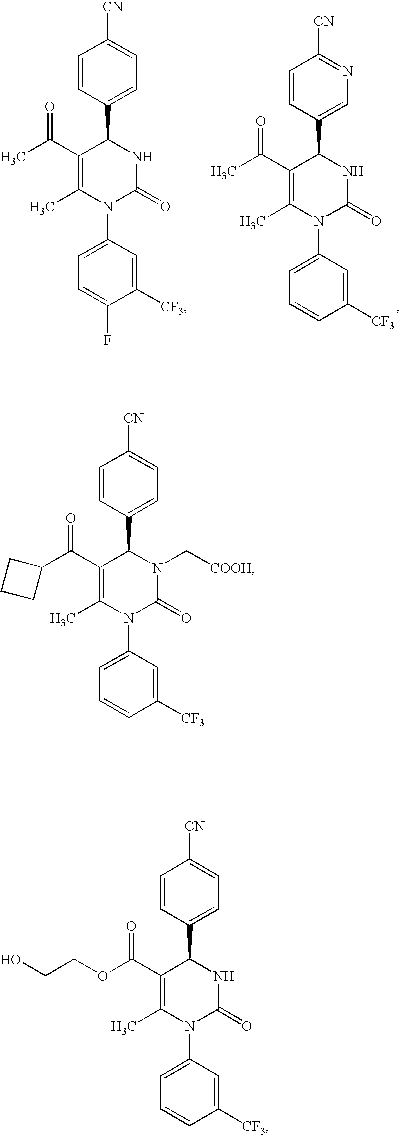 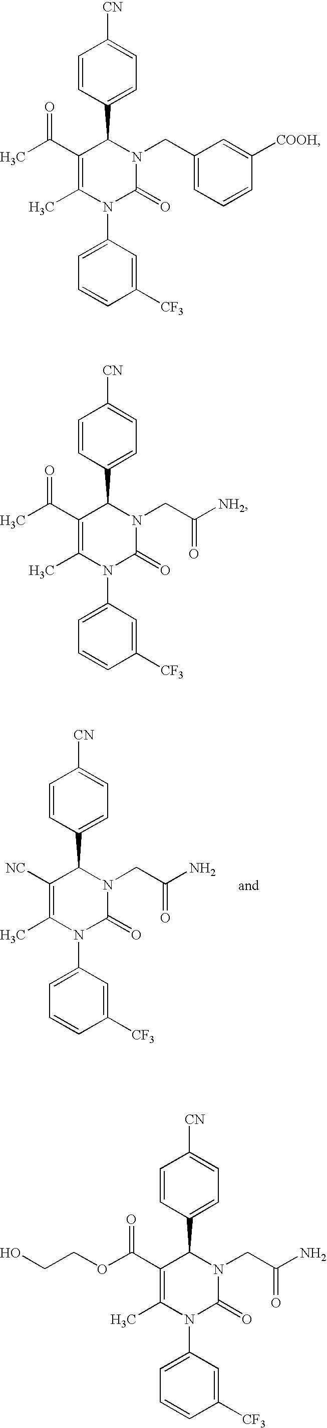
and the salts, solvates and solvates of the salts thereof.

4. The use as claimed in claim 1 of a compound of formula (I) as claimed in claim 1, selected from the group of compounds consisting of 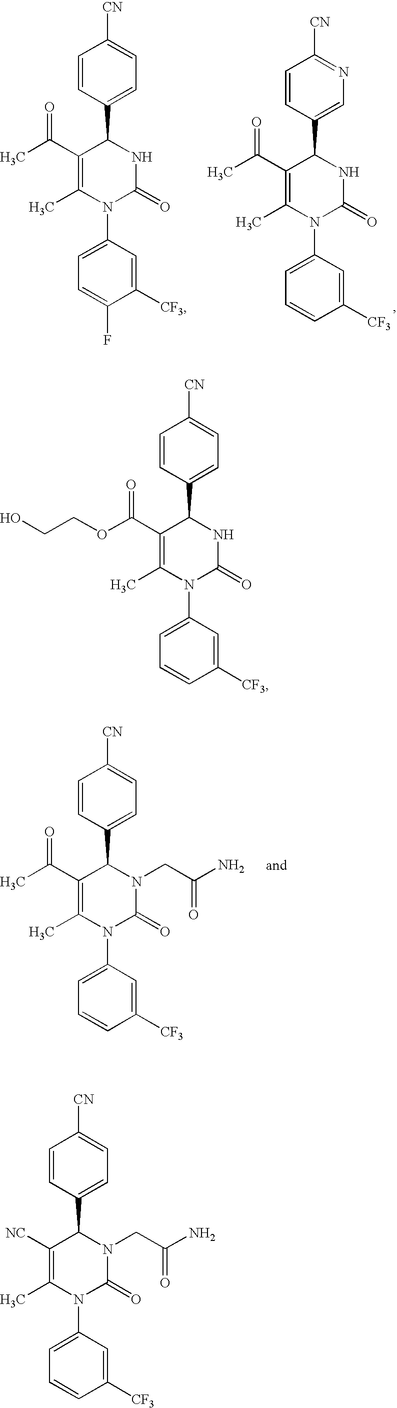
and the salts, solvates and solvates of the salts thereof.

5. A compound of formula (I) as claimed in claim 1 with one of the following structures: 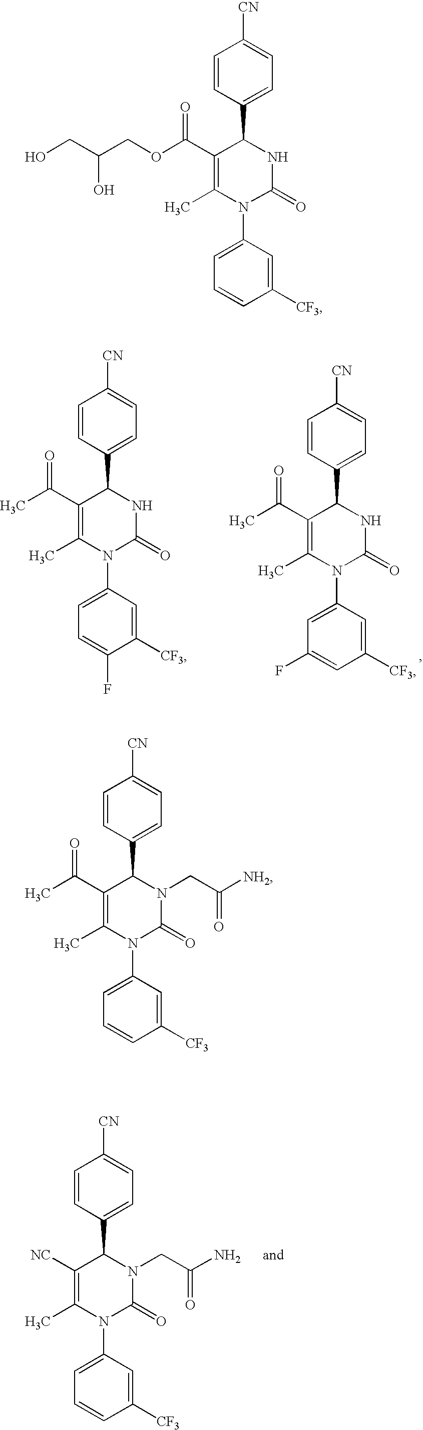 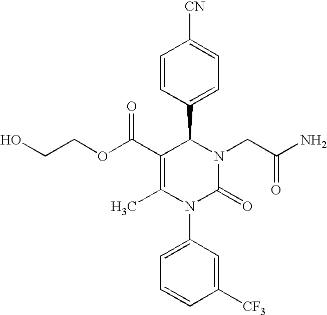
and the salts, solvates and solvates of the salts thereof.

6. A compound as claimed in claim 5 for the treatment and/or prophylaxis of diseases.

7. A pharmaceutical composition comprising at least one compound of the formula (I) as defined in claim 1, and optionally one or more further active ingredients selected from the group consisting of kinase inhibitors, stimulators and activators of soluble guanylate cyclase, prostacyclin analogs, endothelin receptor antagonists and phosphodiesterase inhibitors, and a pharmaceutically acceptable carrier.

8. (canceled)

9. The use of a compound of the formula (I) as defined in claim 1, for the manufacture of a medicament for the treatment and/or prophylaxis of idiopathic or familial pulmonary arterial hypertension, or pulmonary arterial hypertension associated with medicaments, toxins or other disorders, for the treatment and/or prophylaxis of pulmonary hypertension associated with left atrial or left ventricular disorders, left heart valve disorders, chronic obstructive pulmonary disease, interstitial pulmonary disease, pulmonary fibrosis, sleep apnoea syndrome, disorders with alveolar hypoventilation, altitude sickness, pulmonary development impairments, chronic thrombotic and/or embolic disorders or in conjunction with sarcoidosis, histiocytosis X or lymphangioleiomyomatosis, and for the treatment and/or prophylaxis of pulmonary hypertension caused by external compression of vessels.

10. A pharmaceutical composition comprising a compound as defined in claim 5, where appropriate combined with one or more inert, non-toxic, pharmaceutically suitable excipients.

11. (canceled)

12. The pharmaceutical composition as claimed in claim 10 for the treatment and/or prophylaxis of pulmonary arterial hypertension and other types of pulmonary hypertension.

13. A method for the treatment and/or prophylaxis of pulmonary arterial hypertension and other types of pulmonary hypertension in humans and animals by administering an effective amount of at least one compound of the formula (I) as defined in claim 1.

14. A method for the treatment and/or prophylaxis of pulmonary arterial hypertension and other types of pulmonary hypertension in humans and animals by administering an effective amount of a pharmaceutical composition of claim 7.
